SEQUENCE LISTING (38 sequences)

SEQ ID NO: 1 - protein, 17 aa, synthetic construct
KSSQSLLNSRTRKNYLA

SEQ ID NO: 2 - protein, 7 aa, synthetic construct
WASTRES

SEQ ID NO: 3 - protein, 8 aa, synthetic construct
KQSYILRT

SEQ ID NO: 4 - protein, 7 aa, synthetic construct
GYTFTNY

SEQ ID NO: 5 - protein, 6 aa, synthetic construct
YCGDGS

SEQ ID NO: 6 - protein, 10 aa, synthetic construct
DWAGSYFFDF

SEQ ID NO: 7 - protein, 112 aa, synthetic construct
DIQMTQSPSSLSASVGDRVTITCKSSQSLLNSRTRKNYLAWYQQKPGKAPKLLIYWASTRESGVPSRFSGSGSGTDFTFTISSLQPEDIATYYCKQSYILRTFGGGTKVEIK

SEQ ID NO: 8 - protein, 119 aa, synthetic construct
QVQLQESGPGLVKPSETLSLTCTASGYTFTNYYIHWVRQSPGKGLEWIGWIYCGDGSTKFNEQFKDRTTLSADHSKTQASLKLSSVTAADTAIYFCARDWAGSYFFDFWGQGTMVTVSS

SEQ ID NO: 9 - protein, 112 aa, synthetic construct
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYILRTFGGGTKLEIK

SEQ ID NO: 10 - protein, 119 aa, synthetic construct
QVQLQQSGPELVKPGASVRMSCKASGYTFTNYYIHWVRQRPGQGLEWIGWIYCGDGSTKFNEQFKDRTTLTADHSSSTAYMLLSSLTSEDSAIYFCARDWAGSYFFDFWGQGTTLTVSS

SEQ ID NO: 11 - protein, 112 aa, synthetic construct
DIVMSQSPSSLAVSAGEKVTISCKSSQSLLNNRTRKNYLAWYQQKPGQSPKLLIYWASTRESGVPDRFTGSGSGTDFTLTISSVQAEDLAVYYCKQSYILRTFGGGTKLEIK

SEQ ID NO: 12 - protein, 117 aa, synthetic construct
QVQLQQSGPDLVRPGASVKMSCKASGYTFTNYYVHWLNQRPGQGLEWIGWIYPGDGSTKFNEKFKGKTTLTADKSSSTAYILLSSLTSADSAIYFCTRDAGYYFDVWGAGTTVTVSS

SEQ ID NO: 13 - protein, 16 aa, synthetic construct
  MOD_RES 1: note = pyroglutamine
QDGNEEMGGITQTPYK

SEQ ID NO: 14 - protein, 440 aa, synthetic construct
QDGNEEMGSITQTPYQVSISGTTVILTCSQHLGSEAQWQHNGKNKEDSGDRLFLPEFSEMEQSGYYVCYPRGSNPEDASHHLYLKARVCENCMEMDGGGSGGGSGGGSGGGSGGGSQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 15 - protein, 461 aa, synthetic construct
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDGGGSGGGSGGGSGGGSGGGSQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTSYG

SEQ ID NO: 16 - protein, 6 aa, synthetic construct
YSGDGS

SEQ ID NO: 17 - protein, 119 aa, synthetic construct
QVQLQESGPGLVKPSETLSLTCTASGYTFTNYYIHWVRQSPGKGLEWIGWIYSGDGSTKFNEQFKDRTTLSADHSKTQASLKLSSVTAADTAIYFCARDWAGSYFFDFWGQGTMVTVSS

SEQ ID NO: 18 - protein, 222 aa, synthetic construct
QVQLQESGPGLVKPSETLSLTCTASGYTFTNYYIHWVRQSPGKGLEWIGWIYSGDGSTKFNEQFKDRTTLSADHSKTQASLKLSSVTAADTAIYFCARDWAGSYFFDFWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 19 - protein, 6 aa, synthetic construct
YPGDGS

SEQ ID NO: 20 - protein, 8 aa, synthetic construct
DAGYYFDV

SEQ ID NO: 21 - protein, 119 aa, synthetic construct
QVQLQESGPGLVKPSETLSLTCTASGYTFTNYYIFWVRQSPGKGLEWIGWIYCGDGSTKFNEQFKDRTTLSADHSKTQASLKLSSVTAADTAIYFCARDWAGSYFFDFWGQGTMVTVSS

SEQ ID NO: 22 - protein, 222 aa, synthetic construct
QVQLQESGPGLVKPSETLSLTCTASGYTFTNYYIFWVRQSPGKGLEWIGWIYCGDGSTKFNEQFKDRTTLSADHSKTQASLKLSSVTAADTAIYFCARDWAGSYFFDFWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 23 - protein, 119 aa, synthetic construct
QVQLQESGPGLVKPSETLSLTCTASGYTFTNYYIYWVRQSPGKGLEWIGWIYCGDGSTKFNEQFKDRTTLSADHSKTQASLKLSSVTAADTAIYFCARDWAGSYFFDFWGQGTMVTVSS

SEQ ID NO: 24 - protein, 222 aa, synthetic construct
QVQLQESGPGLVKPSETLSLTCTASGYTFTNYYIYWVRQSPGKGLEWIGWIYCGDGSTKFNEQFKDRTTLSADHSKTQASLKLSSVTAADTAIYFCARDWAGSYFFDFWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 25 - protein, 119 aa, synthetic construct
QVQLQESGPGLVKPSETLSLTCTASGYTFTNYYIQWVRQSPGKGLEWIGWIYCGDGSTKFNEQFKDRTTLSADHSKTQASLKLSSVTAADTAIYFCARDWAGSYFFDFWGQGTMVTVSS

SEQ ID NO: 26 - protein, 222 aa, synthetic construct
QVQLQESGPGLVKPSETLSLTCTASGYTFTNYYIQWVRQSPGKGLEWIGWIYCGDGSTKFNEQFKDRTTLSADHSKTQASLKLSSVTAADTAIYFCARDWAGSYFFDFWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 27 - protein, 119 aa, synthetic construct
QVQLQESGPGLVKPSETLSLTCTASGYTFTNYSIHWVRQSPGKGLEWIGWIYCGDGSTKFNEQFKDRTTLSADHSKTQASLKLSSVTAADTAIYFCARDWAGSYFFDFWGQGTMVTVSS

SEQ ID NO: 28 - protein, 222 aa, synthetic construct
QVQLQESGPGLVKPSETLSLTCTASGYTFTNYSIHWVRQSPGKGLEWIGWIYCGDGSTKFNEQFKDRTTLSADHSKTQASLKLSSVTAADTAIYFCARDWAGSYFFDFWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 29 - protein, 220 aa, synthetic construct
DIQMTQSPSSLSASVGDRVTITCKSSQSLLNSRTRKNYLAWYQQKPGKAPKLLIYWASTRESGVPSRFSGSGSGTDFTFTISSLQPEDIATYYCKQSYILRTFGGGTKVEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 222 aa, synthetic construct
QVQLQESGPGLVKPSETLSLTCTASGYTFTNYYIHWVRQSPGKGLEWIGWIYCGDGSTKFNEQFKDRTTLSADHSKTQASLKLSSVTAADTAIYFCARDWAGSYFFDFWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 31 - protein, 219 aa, synthetic construct
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYILRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 222 aa, synthetic construct
QVQLQQSGPELVKPGASVRMSCKASGYTFTNYYIHWVRQRPGQGLEWIGWIYCGDGSTKFNEQFKDRTTLTADHSSSTAYMLLSSLTSEDSAIYFCARDWAGSYFFDFWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 33 - protein, 219 aa, synthetic construct
DIVMSQSPSSLAVSAGEKVTISCKSSQSLLNNRTRKNYLAWYQQKPGQSPKLLIYWASTRESGVPDRFTGSGSGTDFTLTISSVQAEDLAVYYCKQSYILRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 220 aa, synthetic construct
QVQLQQSGPDLVRPGASVKMSCKASGYTFTNYYVHWLNQRPGQGLEWIGWIYPGDGSTKFNEKFKGKTTLTADKSSSTAYILLSSLTSADSAIYFCTRDAGYYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 35 - protein, 105 aa, synthetic construct
QDGNEEMGGITQTPYKYSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 36 - protein, 96 aa, synthetic construct
QDGNEEMGSITQTPYQVSISGTTVILTCSQHLGSEAQWQHNGKNKEDSGDRLFLPEFSEMEQSGYYVCYPRGSNPEDASHHLYLKARVCENCMEMD

SEQ ID NO: 37 - protein, 109 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 38 - protein, 125 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSS

(19) United States
(12) Patent Application Publication
(10) Pub. No.: US 2024/0400686 A1
(43) Pub. Date: Dec. 5, 2024

(54) ANTI-CD3 ANTIBODIES AND USES THEREOF

(71) Applicant: CITY OF HOPE, Duarte, CA (US)

(72) Inventors: John C. WILLIAMS, Monrovia, CA (US); Alfur HUNG, Duarte, CA (US); Miso PARK, Duarte, CA (US)

(21) Appl. No.: 18/697,236

(22) PCT Filed: Sep. 29, 2022
(86) PCT No.: PCT/US2022/077304

§ 371 Date: Mar. 29, 2024

Related U.S. Application Data
(60) Provisional application No. 63/250,855, filed on Sep. 30, 2021.

(51) Int. Cl. C07K 16/28

(52) U.S. Cl. CPC C07K 16/2809; C07K 2317/24; C07K 2317/33; C07K 2317/622; C07K 2317/92

(57) ABSTRACT

Provided herein are, inter alia, antibodies capable of binding human and non-human primate (e.g., cynomolgus) CD3. The anti-CD3 antibodies provided herein including embodiments thereof may be used for therapeutic purposes, for example, as humanized anti-CD3 antibodies, or they may form part of bispecific antibodies. Further provided herein are, inter alia, non-CD3 binding antibodies that are derived from the anti-CD3 antibodies provided herein including embodiments thereof, and which due to the presence of amino acid substitutions have lost their ability to bind to CD3.

### RELATED APPLICATION DATA

This application claims the benefit of priority under 35 U.S.C. § 119(e) of the U.S. Patent Application No. 63/250,855, filed on Sep. 30, 2021, which is hereby incorporated by reference in its entirety and for all purposes.

### SEQUENCE LISTING

The material in the accompanying Sequence Listing is hereby incorporated by reference in its entirety. The accompanying file, named “048440-818001WO_SL_ST26.xml” was created on Sep. 28, 2022 on a computer that uses Windows OS and is 38,905 bytes.

### BACKGROUND OF THE INVENTION

There is a need in the art for anti-CD3 antibodies that bind and activate CD3 expressing T cells and are therefore useful for cancer therapeutic purposes. Provided herein are antibody compositions and methods that address this and other needs in the art.

### BRIEF SUMMARY OF THE INVENTION

In a first aspect is provided an anti-CD3 antibody including a light chain variable domain and a heavy chain variable domain, wherein the light chain variable domain includes: a CDR L1 as set forth in SEQ ID NO: 1, a CDR L2 as set forth in SEQ ID NO:2 and a CDR L3 as set forth in SEQ ID NO: 3; and wherein the heavy chain variable domain includes: a CDR H1 as set forth in SEQ ID NO:4, a CDR H2 as set forth in SEQ ID NO:5, and a CDR H3 as set forth in SEQ ID NO: 6.

In another aspect is provided an anti-CD3 antibody including a light chain variable domain and a heavy chain variable domain, wherein the light chain variable domain includes: a CDR L1 as set forth in SEQ ID NO: 1, a CDR L2 as set forth in SEQ ID NO:2 and a CDR L3 as set forth in SEQ ID NO: 3; and wherein the heavy chain variable domain includes: a CDR H1 as set forth in SEQ ID NO:4, a CDR H2 as set forth in SEQ ID NO:16, and a CDR H3 as set forth in SEQ ID NO: 6.

In another aspect is provided an anti-CD3 antibody including a light chain variable domain and a heavy chain variable domain, wherein the light chain variable domain includes: a CDR L1 as set forth in SEQ ID NO:1, a CDR L2 as set forth in SEQ ID NO:2 and a CDR L3 as set forth in SEQ ID NO: 3; and wherein the heavy chain variable domain includes: a CDR H1 as set forth in SEQ ID NO: 4, a CDR H2 as set forth in SEQ ID NO:19, and a CDR H3 as set forth in SEQ ID NO: 20.

In another aspect is provided a recombinant protein including: (i) a first antibody region capable of binding a target cell ligand; and (ii) a second antibody region, including: (a) a second light chain variable domain including a CDR L1 as set forth in SEQ ID NO:1, a CDR L2 as set forth in SEQ ID NO:2 and a CDR L3 as set forth in SEQ ID NO: 3; and (b) a second heavy chain variable domain including a CDR H1 as set forth in SEQ ID NO:4, a CDR H2 as set forth in SEQ ID NO:5, and a CDR H3 as set forth in SEQ ID NO: 6.

In another aspect is provided a recombinant protein including (i) a first antibody region capable of binding a target cell ligand; and (ii) a second antibody region, including: (a) a second light chain variable domain including a CDR L1 as set forth in SEQ ID NO:1, a CDR L2 as set forth in SEQ ID NO:2 and a CDR L3 as set forth in SEQ ID NO: 3; and (b) a second heavy chain variable domain including a CDR H1 as set forth in SEQ ID NO:4, a CDR H2 as set forth in SEQ ID NO:16, and a CDR H3 as set forth in SEQ ID NO: 6.

In another aspect is provided a recombinant protein including (i) a first antibody region capable of binding a target cell ligand; and (ii) a second antibody region, including: (a) a second light chain variable domain including a CDR L1 as set forth in SEQ ID NO:1, a CDR L2 as set forth in SEQ ID NO:2 and a CDR L3 as set forth in SEQ ID NO: 3; and (b) a second heavy chain variable domain including a CDR H1 as set forth in SEQ ID NO:4, a CDR H2 as set forth in SEQ ID NO:19, and a CDR H3 as set forth in SEQ ID NO:20.

In another aspect is provided a pharmaceutical composition including (i) a therapeutically effective amount of an anti-CD3 antibody as provided herein including embodiments thereof, or a therapeutically effective amount of a recombinant protein as provided herein including embodiments thereof and (ii) a pharmaceutically acceptable excipient.

In another aspect is provided a method of treating cancer in a subject in need thereof, the method including administering to a subject a therapeutically effective amount of an anti-CD3 antibody as provided herein including embodiments thereof or a therapeutically effective amount of a recombinant protein as provided herein including embodiments thereof, thereby treating cancer in the subject.

In an aspect is provided a method of treating a disease in a subject in need thereof, the method including administering to a subject a therapeutically effective amount of an anti-CD3 antibody as provided herein including embodiments thereof, or a therapeutically effective amount of a recombinant protein as provided herein including embodiments thereof, thereby treating the disease in the subject.

In another aspect is provided a isolated nucleic acid encoding an anti-CD3 antibody as provided herein including embodiments thereof.

In another aspect is provided a isolated nucleic acid encoding a recombinant protein as provided herein including embodiments thereof.

In another aspect is provided a cell including an anti-CD3 antibody as provided herein including embodiments thereof.

In another aspect is provided a cell including a recombinant protein as provided herein including embodiments thereof.

In another aspect is provided a method of forming an anti-CD3 antibody, the method including administering a polypeptide including the sequence of SEQ ID NO:14 or SEQ ID NO:15 to a mammal, thereby forming an anti-CD3 antibody.

### BRIEF DESCRIPTION OF THE DRAWINGS

FIG. 1 illustrates construction of CD3 antigens for mouse immunization and hybridoma screening.

FIG. 2 illustrates CD3 protein analysis with a size-exclusion column.

FIG. 3 illustrates antibody ELISA screening with hybridoma culture supernatants.

FIG. 4 illustrates binding epitope analysis of identified anti-CD3 mAbs.

FIG. 5 illustrates flow cytometry analysis of identified anti-CD3 mAbs.

FIG. 6 illustrates flow cytometry analysis of CD69 expression on Jurkat cells treated with anti-CD3 mAbs.

FIG. 7 illustrates isotyping with ELISA for anti-CD3 mAb.

FIG. 8 illustrates PCR-amplified fragments for VH and VL cloning of anti-CD3 mAbs.

FIG. 9 illustrates flow cytometric analysis of CD69 expression for human PBMCs stimulated with anti-CD3 mAbs.

FIG. 10 illustrates proliferation assays for human PBMCs treated with anti-CD3 mAbs.

FIG. 11 illustrates cytokine ELISA for human PBMCs treated with anti-CD3 mAbs.

FIG. 12 illustrates binding affinity measurement for anti-CD3 mAbs.

FIG. 13 illustrates binding affinity measurement for Humanized anti-CD3, 26H8 Fab.

FIG. 14 illustrates Protein Thermal Shift for Humanized anti-CD3, 26H8 Fab.

FIG. 15 illustrates a model of N-terminal peptide bound to h26H8 Fab. Depicted is a sticks diagram. The Fab and peptide were mixed in 1:1 molar ratio and concentrated to 6 mg/ml in 25 mM Tris, 50 ml NaCl buffer. Crystals were obtained by vapor diffusion as sitting drops, mixing 1 part CD3 fab/peptide complex with 1 part mother liquor. The mother liquor was 0.1M HEPES: NaOH, pH 7.5, 10% (w/v) PEG 8000, 8% (v/v) Ethylene Glycol. Diffraction data was collected at Stanford Synchrotron Radiation Lightsource and the diffraction images were reduced using XDS. The structure was determined using molecular replacement and refined, in an iterative process using manual building using Coot and refinement protocols as implemented in Phenix. See also Table 1.

FIG. 16 illustrates Surface Diagram showing that the pyroglutamate is buried between the light and heavy chains of the Fab.

FIG. 17 illustrates Electrostatic Surface with N-terminal CD3 sticks. Mapping the electrostatics on to the surface shows the peptide interacts with a positively charged region on the Fab. Note the peptide is negatively charged with one aspartate and two glutamates (pyro-QDGNEEMGG).

FIG. 18 illustrates ligplot of CD3 N-terminus bound to h26h8. Interactions between the N-terminal peptide (L) and Fab residues (B—light chain, C—heavy chain) are depicted in the Ligplot diagram. Note the pyroglutamate is buried between the CDR loops, making a hydrogen bond to His-35 (C) and van der Waals contacts to Tyr-38(B). In addition, there is an ionic interaction between the guanidium of Arg101 and backbone carbonyl of the CD3 pyroglutamate (Pyro-1). Other interactions include the carboxylate of Asp-2 to the amide backbone of Leu-100 (B); backbone/backbone between Gly-3 (L) and Ser-97(B); backbone carbonyls of Gly-3(L) and Glu-6 (L) to the side chain N of Asn-31(B); backbone carbonyl of Tyr-98(B) to the backbone of Asn-4 (L); backbone carbonyls of Asn-4 (L) and Glu-6 (L) to the guanidinium of Arg-33 (B). Finally, there are number of van der Waals interactions from both the heavy and light chains. Notably, many of the direct electrostatic interactions are between the light chain CDR3 and the peptide.

FIG. 19 illustrates CDR loops h26h8. The CDR loops of the light and heavy chains are mapped on the h26H8 model.

FIG. 20 illustrates the sequence alignment of human (SEQ ID NO:35) CD3 episilon (top) and cynomolgus monkey (SEQ ID NO:36) CD3 episilon (bottom). The first eight residues are identical as shown in the sequence alignment. Critically, the first residue is a glutamine and naturally undergoes a cyclization reaction to form a lactam. This is denoted in the sequence as pyro-Q. The molecular structure of the lactam is shown directly below the sequence. The peptide containing the first eight (identical) sequences was synthesized (SEQ ID NO:13). This peptide was then co-crystalized with 26H8 Fab and diffraction studies were conducted. The structure obtained shows the N-terminal peptide between the heavy and light chains making a number of electrostatic and hydrophobic contacts. Based on this structure, several residues were identified whose mutation are expected to reduce/vitiate binding to the CD3 epsilon peptide, but not significantly alter the structure, thereby provide negative control. These mutations were generated, expressed, purified and tested using surface plasmon resonance.

FIGS. 21A-21E illustrate purification and characterization of anti-CD3 Fab′ clones provided herein. FIGS. 21A-21D show size-exclusion chromatography of anti-CD3 antibody Fab′ clones h26H8 H35F (FIG. 21A), h26H8 H35Y (FIG. 21B), h26H3 H35Q (FIG. 21C) and h26H8 Y33S (FIG. 21D). FIG. 21E shows pictures of SDS-PAGE blots with anti-CD3 Fab′ clones.

FIGS. 22A-22E illustrate binding of anti-CD3 h26H8 Fab′ clones provided herein to a CD3 peptide on surface plasmon resonance (SPR).

FIG. 23 illustrates thermal shift curves of anti-CD3 h26H8 Fab′ clones provided herein.

FIG. 24 illustrates size-exclusion chromatography of h26H8 C53S Fab′ provided herein.

FIGS. 25A-25C illustrate binding of anti-CD3 h26H8 Fab′ C53S (FIG. 25A) and h26H8 Fab′ (FIG. 25B) provided herein to a CD3 peptide on surface plasmon resoanance (SPR). FIG. 25C shows quantification of the SPR data for h26H8 Fab′ C53S and h26H8 Fab′ clones provided herein.

### DETAILED DESCRIPTION OF THE INVENTION

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amio acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that may be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein (e.g., anti-CD3 antibody) in other proteins with different numbering systems. For example, by performing a simple sequence alignment with a protein (e.g., CD3 protein) the identity and location of residues corresponding to specific positions of the protein are identified in other protein sequences aligning to the protein. For example, a selected residue in a selected protein corresponds to serine at position 53 when the selected residue occupies the same essential spatial or other structural relationship as a serine at position 53. In some embodiments, where a selected protein is aligned for maximum homology with a protein, the position in the aligned selected protein aligning with serine 53 is the residue to correspond to serine 53. Instead of a primary sequence alignment, a three dimensional structural alignment can also be used, e.g., where the structure of the selected protein is aligned for maximum correspondence with the serine at position 53, and the overall structures compared. In this case, an amino acid that occupies the same essential position as serine 53 in the structural model is the residue to correspond to the serine 53 residue.

Likewise, a selected residue in a selected antibody (or Fab domain) corresponds to light chain threonine at Kabat position 40, when the selected residue occupies the same essential spatial or other structural relationship as a light chain threonine at Kabat position 40. In some embodiments, where a selected protein is aligned for maximum homology with the light chain of an antibody (or Fab domain), the position in the aligned selected protein aligning with threonine 40 is said to correspond to threonine 40. Instead of a primary sequence alignment, a three dimensional structural alignment can also be used, e.g., where the structure of the selected protein is aligned for maximum correspondence with the light chain threonine at Kabat position 40, and the overall structures compared. In this case, an amino acid that occupies the same essential position as threonine 40 in the structural model is said to correspond to the threonine 40 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region, involved in binding the target antigen, and a constant (“C”) region that interacts with other components of the immune system. The light and heavy chain variable regions include domains (also referred to herein as light chain variable (VL) domain and heavy chain variable (VH) domain, respectively) that come together in 3-dimensional space to form a variable region that binds the antigen (for example, a receptor on the surface of a cell). Within each light or heavy chain variable domain, there are three short segments (averaging 10 amino acids in length) called the complementarity determining regions (“CDRs”). The six CDRs in an antibody variable region (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An “antibody variant” as provided herein refers to a polypeptide capable of binding to an antigen and including one or more structural domains of an antibody or fragment thereof. Non-limiting examples of antibody variants include single-domain antibodies or nanobodies, affibodies (polypeptides smaller than monoclonal antibodies (e.g., about 6 kDA) and capable of binding antigens with high affinity and imitating monoclonal antibodies, monospecific Fab₂, bispecific Fab₂, trispecific Fab₃, monovalent IgGs, scFv, bispecific diabodies, trispecific triabodies, scFv-Fc, minibodies, IgNAR, V-NAR, hcIgG, VhH, or peptibodies. A “nanobody” or “single domain antibody” as described herein is commonly well known in the art and refers to an antibody fragment consisting of a single monomeric variable antibody domain. Like a whole antibody, it is able to bind selectively to a specific antigen. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805, which are incorporated, by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

The term “bispecific T-cell engager (BiTE)”, “BiTe” or “bispecific antibody” as provided herein is used according to its conventional meaning well known in the art and refers to a bispecific recombinant protein capable to simultaneously bind to two different antigens. In contrast to traditional monoclonal antibodies, BiTE antibodies consist of two independently different antibody regions (e.g., two single-chain variable fragments (scFv)), each of which binds a different antigen. One antibody region engages effector cells (e.g., T cells) by binding an effector cell-specific antigen (e.g., CD3 molecule) and the second antibody region binds a target cell (e.g., cancer cell or autoimmune-reactive cell) through a cell surface antigen (e.g., cancer protein) expressed by said target cell. Binding of the BiTE to the two antigens will link the effector cell (e.g., T cell) to the target cell (e.g., tumor cell) and activate the effector cell (e.g., T cell) via effector cell-specific antigen signaling (e.g., CD3 signaling). The activated effector cell (e.g., T cell) will then exert cytotoxic activity against the target cell (e.g., tumor cells).

The terms “CDR L1”, “CDR L2” and “CDR L3” as provided herein refer to the complementarity determining regions (CDR) 1, 2, and 3 of the variable light (L) chain of an antibody. In embodiments, the variable light chain provided herein includes in N-terminal to C-terminal direction a CDR L1, a CDR L2 and a CDR L3. Likewise, the terms “CDR H1”, “CDR H2” and “CDR H3” as provided herein refer to the complementarity determining regions (CDR) 1, 2, and 3 of the variable heavy (H) chain of an antibody. In embodiments, the variable heavy chain provided herein includes in N-terminal to C-terminal direction a CDR H1, a CDR H2 and a CDR H3. In embodiments, the CDRs of the light chain are referred to as CDR1, CDR2, and CDR3 of VL and the CDRs of the heavy chain are referred to as CDR1, CDR2, and CDR3 of VH. See, for example the tables as provided herein.

The terms “FR L1”, “FR L2”, “FR L3” and “FR L4” as provided herein are used according to their common meaning in the art and refer to the framework regions (FR) 1, 2, 3 and 4 of the variable light (L) chain of an antibody. In embodiments, the variable light chain provided herein includes in N-terminal to C-terminal direction a FR L1, a FR L2, a FR L3 and a FR L4. Likewise, the terms “FR H1”, “FR H2”, “FR H3” and “FR H4” as provided herein are used according to their common meaning in the art and refer to the framework regions (FR) 1, 2, 3 and 4 of the variable heavy (H) chain of an antibody. In embodiments, the variable heavy chain provided herein includes in N-terminal to C-terminal direction a FR H1, a FR H2, a FR H3 and a FR H4.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL), variable light chain (VL) domain or light chain variable domain and variable heavy chain (VH), variable heavy chain (VH) domain or heavy chain variable domain refer to these light and heavy chain regions, respectively. The terms variable light chain (VL), variable light chain (VL) domain and light chain variable domain as referred to herein may be used interchangeably. The terms variable heavy chain (VH), variable heavy chain (VH) domain and heavy chain variable domain as referred to herein may be used interchangeably. The Fc (i.e. fragment crystallizable region) is the “base” or “tail” of an immunoglobulin and is typically composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985)). “Monoclonal” antibodies (mAb) refer to antibodies derived from a single clone. Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL), variable light chain (VL) domain or light chain variable region and variable heavy chain (VH), variable heavy chain (VH) domain or heavy chain variable region refer to these light and heavy chain regions, respectively. The terms variable light chain (VL), variable light chain (VL) domain and light chain variable region as referred to herein may be used interchangeably. The terms variable heavy chain (VH), variable heavy chain (VH) domain and heavy chain variable region as referred to herein may be used interchangeably. The Fc (i.e. fragment crystallizable region; also referred to herein as “Fc domain”) is the “base” or “tail” of an immunoglobulin and is typically composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins. In embodiments, the Fc region includes a constant heavy chain domain 3 (CH3 domain) and a constant heavy chain domain 2 (CH2 domain).

The epitope of an antibody is the region of its antigen to which the antibody binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

The term “antigen” as provided herein refers to molecules capable of binding to the antibody binding domain provided herein. An “antigen binding domain” as provided herein is a region of an antibody that binds to an antigen (epitope). As described above, the antigen binding domain is generally composed of one constant and one variable domain of each of the heavy and the light chain (VL, VH, CL and CH1, respectively). The paratope or antigen-binding site is formed on the N-terminus of the antigen binding domain. The two variable domains of an antigen binding domain typically bind the epitope on an antigen.

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa. In embodiments, the linker includes more than one serine. In embodiments, the linker includes more than one glycine. In embodiments, the linker has the structure of -(Gly-Gly-Gly-Gly-Ser)₃-.

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

A “ligand” refers to an agent, e.g., a polypeptide or other molecule, capable of binding to a receptor or antibody, antibody variant, antibody region or fragment thereof.

Techniques for conjugating therapeutic agents to antibodies are well known (see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2^{nd} Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)). As used herein, the term “antibody-drug conjugate” or “ADC” refers to a therapeutic agent conjugated or otherwise covalently bound to to an antibody.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

The term “CD3” or “CD3 protein” as used herein refers to any recombinant or naturally-occurring forms of Cluster of Differentiation 3 (CD3) or variants, isoforms or homologs thereof that maintain CD3 activity (e.g. within at least 50%, 80%, 90%, 95%, 96%, 97%, 98%, 99% or 100% activity compared to CD3). In some aspects, the variants, isoforms or homologs have at least 90%, 95%, 96%, 97%, 98%, 99% or 100% amino acid sequence identity across the whole sequence or a portion of the sequence (e.g. a 10, 20, 50, 100, 150 or 200 continuous amino acid portion) compared to a naturally occurring CD3 polypeptide. In embodiments, CD3 is substantially identical to the protein identified by the UniProt reference number P07766 or a variant, isoform or homolog having substantial identity thereto. In embodiments, CD3 is substantially identical to the protein identified by the UniProt reference number P09693 or a variant, isoform or homolog having substantial identity thereto. In embodiments, CD3 is substantially identical to the protein identified by the UniProt reference number P04234 or a variant, isoform or homolog having substantial identity thereto.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The terms “plasmid”, “vector” or “expression vector” refer to a nucleic acid molecule that encodes for genes and/or regulatory elements necessary for the expression of genes. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

When the label or detectable moiety is a radioactive metal or paramagnetic ion, the agent may be reacted with another long-tailed reagent having a long tail with one or more chelating groups attached to the long tail for binding to these ions. The long tail may be a polymer such as a polylysine, polysaccharide, or other derivatized or derivatizable chain having pendant groups to which the metals or ions may be added for binding. Examples of chelating groups that may be used according to the disclosure include, but are not limited to, ethylenediaminetetraacetic acid (EDTA), diethylenetriaminepentaacetic acid (DTPA), DOTA, NOTA, NETA, TETA, porphyrins, polyamines, crown ethers, bis-thiosemicarbazones, polyoximes, and like groups. The chelate is normally linked to the PSMA antibody or functional antibody fragment by a group, which enables the formation of a bond to the molecule with minimal loss of immunoreactivity and minimal aggregation and/or internal cross-linking. The same chelates, when complexed with non-radioactive metals, such as manganese, iron and gadolinium are useful for MRI, when used along with the antibodies and carriers described herein. Macrocyclic chelates such as NOTA, DOTA, and TETA are of use with a variety of metals and radiometals including, but not limited to, radionuclides of gallium, yttrium and copper, respectively. Other ring-type chelates such as macrocyclic polyethers, which are of interest for stably binding nuclides, such as ²²³Ra for RAIT may be used. In certain embodiments, chelating moieties may be used to attach a PET imaging agent, such as an Al-¹⁸F complex, to a targeting molecule for use in PET analysis.

“Contacting” is used in accordance with its plain ordinary meaning and refers to the process of allowing at least two distinct species (e.g. antibodies and antigens) to become sufficiently proximal to react, interact, or physically touch. It should be appreciated; however, that the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, a pharmaceutical composition as provided herein and a cell. In embodiments, contacting includes, for example, allowing a pharmaceutical composition as described herein to interact with a cell.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include, but are not limited to, yeast cells and cells derived from plants and animals, for example mammalian, insect (e.g., spodoptera) and human cells.

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism.

As defined herein, the term “inhibition”, “inhibit”, “inhibiting” and the like in reference to cell proliferation (e.g., cancer cell proliferation) means negatively affecting (e.g., decreasing proliferation) or killing the cell. In some embodiments, inhibition refers to reduction of a disease or symptoms of disease (e.g., cancer, cancer cell proliferation). Thus, inhibition includes, at least in part, partially or totally blocking stimulation, decreasing, preventing, or delaying activation, or inactivating, desensitizing, or down-regulating signal transduction or enzymatic activity or the amount of a protein. Similarly an “inhibitor” is a compound or protein that inhibits a receptor or another protein, e.g., by binding, partially or totally blocking, decreasing, preventing, delaying, inactivating, desensitizing, or down-regulating activity (e.g., a receptor activity or a protein activity).

Thus, the terms “inhibitor,” “repressor” or “antagonist” or “downregulator” interchangeably refer to a substance capable of detectably decreasing the expression or activity of a given gene or protein. The antagonist can decrease gene or protein expression or activity 10%, 20%, 30%, 40%, 50%, 60%, 70%, 80%, 90% or more in comparison to a control in the absence of the antagonist. In certain instances, gene or protein expression or activity is 1.5-fold, 2-fold, 3-fold, 4-fold, 5-fold, 10-fold or lower than the expression or activity in the absence of the antagonist.

As defined herein, the term “activation”, “activate”, “activating”, “activator” and the like in reference to a cell (e.g., T cell)-ligand interaction means positively affecting (e.g. increasing) the activity or function of the cell relative to the activity or function of the cell in the absence of the ligand. In aspects activation means positively affecting (e.g. increasing) the proliferation rate or biologic activity of the cell relative to the rate or activity of the cell in the absence of the activator. The terms may reference activation, or activating, sensitizing, or up-regulating signal transduction or enzymatic activity or gene expression of a cell. Thus, activation may include, at least in part, partially or totally increasing stimulation, increasing or enabling activation, or activating, sensitizing, or up-regulating signal transduction or enzymatic activity or gene expression relative to the absence of the activator. Activation may include, at least in part, partially or totally increasing stimulation, increasing or enabling activation, or activating, sensitizing, or up-regulating signal transduction or enzymatic activity or gene expression.

The terms “agonist,” “activator,” “upregulator,” etc. refer to a substance capable of detectably increasing the activity or proliferation of a given cell. The agonist can increase activity or proliferation by 10%, 20%, 30%, 40%, 50%, 60%, 70%, 80%, 90% or more in comparison to a control in the absence of the agonist. In aspects, proliferation or activity is 1.5-fold, 2-fold, 3-fold, 4-fold, 5-fold, 10-fold or higher than the proliferation or activity in the absence of the agonist.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “control” or “standard control” refers to a sample, measurement, or value that serves as a reference, usually a known reference, for comparison to a test sample, measurement, or value. For example, a test sample can be taken from a patient suspected of having a given disease (e.g. cancer) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, synoviocytes, synovial fluid, synovial tissue, fibroblast-like synoviocytes, macrophagelike synoviocytes, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

“Patient” or “subject in need thereof” refers to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with the compounds or methods provided herein. The disease may be a cancer. In some further instances, “cancer” refers to human cancers and carcinomas, sarcomas, adenocarcinomas, lymphomas, leukemias, including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including acute myeloid leukemia (AML), ALL, and CML), or multiple myeloma.

As used herein, the term “cancer” refers to all types of cancer, neoplasm or malignant tumors found in mammals (e.g., humans), including leukemia, carcinomas and sarcomas. Exemplary cancers that may be treated with a compound or method provided herein include breast cancer, colon cancer, kidney cancer, leukemia, lung cancer, melanoma, ovarian cancer, prostate cancer, pancreatic cancer, brain cancer, liver cancer, gastric cancer or a sarcoma.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute myeloid leukemia, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatinifori carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The term “associated” or “associated with” in the context of a substance or substance activity or function associated with a disease (e.g., cancer (e.g. lung cancer, triple negative breast cancer, pancreatic cancer)) means that the disease (e.g., cancer (e.g. lung cancer, triple negative breast cancer, pancreatic cancer)) is caused by (in whole or in part), or a symptom of the disease is caused by (in whole or in part) the substance or substance activity or function. Alternatively, the substance may be an indicator of the disease (e.g., cancer (e.g. lung cancer, triple negative breast cancer, pancreatic cancer)). Thus, an associated substance may serve as a means of targeting disease tissue (e.g., cancer cells (e.g., lung cancer, triple negative breast cancer, pancreatic cancer)).

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by using a method as described herein), results in reduction of the disease or one or more disease symptoms.

A “therapeutic agent” as referred to herein, is a composition useful in treating or preventing a disease such as cancer (e.g., leukemia). In embodiments, the therpaeutic agent is an anti-cancer agent. “Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In embodiments, an anti-cancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer.

A “target cell ligand” as provided herein refers to a protein expressed on a cell intended to be bound by the recombinant proteins provided herein including embodiments thereof. The recombinant proteins provide herein may be used for cancer therapeutic purposes. For example, a bispecific antibody may include an anti-CD3 antibody region and a a cancer protein binding region. Therefore, the target cell ligand may be a cancer protein (i.e., a protein expressed by a cancer cell). A “cancer protein” as referred to herein is a protein whose expression is characteristic of a cancer cell. A cancer protein may only be expressed by a cancer cell and not by a healthy cell; it may be expressed at a higher or lower level compared to a healthy cell. A cancer protein may be an aberrant version of a healthy protein.

As used herein, “treating” or “treatment of” a condition, disease or disorder or symptoms associated with a condition, disease (e.g., cancer, e.g., lung cancer, triple negative breast cancer, pancreatic cancer) or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently. As used herein the terms treatment, treat, or treating refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

By “therapeutically effective dose or amount” as used herein is meant a dose that produces effects for which it is administered (e.g. treating or preventing a disease). The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of at least 5%, 1, 1%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease. A therapeutically effective dose or amount may prevent or delay the onset of a disease or one or more symptoms of a disease when the effect for which it is being administered is to treat a person who is at risk of developing the disease.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

In embodiments, the method further includes administering to the subject an additional therapeutic agent. As described above, a therapeutic agent is a composition useful in treating or preventing a disease such as cancer. In embodiments, the additional therapeutic agent is an anti-cancer agent.

The terms “anti-cancer agent” and “anticancer agent” are used in accordance with their plain ordinary meaning and refer to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras famesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or rlL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin A1 (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, Inanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta medica), D-68144 (Asta medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta medica), D-68836 (Asta medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to 111In, 90Y, or 131I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™) afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Antibody Compositions

Provided herein are, inter alia, antibodies capable of binding human and non-human primate CD3 (e.g., cynomolgus CD3). The antibodies provided herein include novel light chain and heavy chain sequences and bind the N-terminal region of CD3 (e.g., CD3ε, CD3γ) with high efficiency and specificity. The anti-CD3 antibodies provided herein including embodiments thereof exhibit improved binding affinity and specificity compared to known anti CD3 antibodies and most importantly have the benefit of being cross-reactive towards human and non-human primate CD3 (e.g, cynomolgus CD3), thereby facilitating production and pre-clinical testing. The anti-CD3 antibodies provided herein including embodiments thereof are, inter alia, useful for targeting and activating T cells. Upon binding of the anti-CD3 antibody provided herein including embodiments thereof to the CD3 protein expressed on an effector cell, the effector cell is activated and able to exert its function (e.g., selective killing or eradication of malignant, infected or otherwise unhealthy cells). The effector cell may be a cytotoxic T cell, a helper T cell, a B cell, or a natural killer cell. The anti-CD3 antibodies provided herein including embodiments thereof, may be used for diagnostic and therapeutic purposes, for example, as humanized anti-CD3 antibodies or they may form part of bispecific antibodies.

Further provided herein are non-CD3 binding antibodies which are derived from the anti-CD3 antibodies provided herein and which include novel light chain and heavy chain sequences. The non-CD3 binding antibodies include light chain and heavy chain sequences derived from the anti-CD3 antibodies provided herein and include amino acid substitions at certain positions. The presence of these substitutions prevents the antibodies from binding to CD3 (e.g., CD3ε, CD3γ) compared to antibodies lacking said substitions. The non-CD3 binding antibodies may, inter alia, be used as test reagents to determine binding specificity of the anti-CD3 antibodies provided herein including embodiments thereof.

In a first aspect is provided an anti-CD3 antibody including a light chain variable domain and a heavy chain variable domain, wherein the light chain variable domain includes: a CDR L1 as set forth in SEQ ID NO: 1, a CDR L2 as set forth in SEQ ID NO:2 and a CDR L3 as set forth in SEQ ID NO: 3; and wherein the heavy chain variable domain includes: a CDR H1 as set forth in SEQ ID NO: 4, a CDR H2 as set forth in SEQ ID NO:5, and a CDR H3 as set forth in SEQ ID NO: 6.

In another aspect is provided an anti-CD3 antibody including a light chain variable domain and a heavy chain variable domain, wherein the light chain variable domain includes: a CDR L1 as set forth in SEQ ID NO: 1, a CDR L2 as set forth in SEQ ID NO:2 and a CDR L3 as set forth in SEQ ID NO: 3; and wherein the heavy chain variable domain includes: a CDR H1 as set forth in SEQ ID NO: 4, a CDR H2 as set forth in SEQ ID NO:16, and a CDR H3 as set forth in SEQ ID NO: 6.

In another aspect is provided an anti-CD3 antibody including a light chain variable domain and a heavy chain variable domain, wherein the light chain variable domain includes: a CDR L1 as set forth in SEQ ID NO: 1, a CDR L2 as set forth in SEQ ID NO:2 and a CDR L3 as set forth in SEQ ID NO: 3; and wherein the heavy chain variable domain includes: a CDR H1 as set forth in SEQ ID NO:4, a CDR H2 as set forth in SEQ ID NO:19, and a CDR H3 as set forth in SEQ ID NO:20.

As described above, a “light chain variable (VL) domain” as provided herein refers to the variable region of the light chain of an antibody, an antibody variant or fragment thereof. Likewise, the “heavy chain variable (VH) domain” as provided herein refers to the variable region of the heavy chain of an antibody, an antibody variant or fragment thereof. The light chain variable domain and the heavy chain variable domain together form the paratope, which binds an antigen (epitope). The paratope or antigen-binding site is formed at the N-terminus of an antibody, an antibody variant or fragment thereof. In embodiments, the light chain variable (VL) domain includes CDR L1, CDR L2, CDR L3 and FR L1, FR L2, FR L3 and FR L4 (framework regions) of an antibody light chain. In embodiments, the heavy chain variable (VH) domain includes CDR H1, CDR H2, CDR H3 and FR H1, FR H2, FR H3 and FR H4 (framework regions) of an antibody heavy chain. In embodiments, the light chain variable (VL) domain and a light chain constant (CL) domain form part of an antibody light chain. In embodiments, the heavy chain variable (VH) domain and a heavy chain constant (CH1) domain form part of an antibody heavy chain. In embodiments, the heavy chain variable (VH) domain and one or more heavy chain constant (CH1, CH2, or CH3) domains form part of an antibody heavy chain. Thus, in embodiments, the light chain variable (VL) domain forms part of an antibody. In embodiments, the heavy chain variable (VH) domain forms part of an antibody. In embodiments, the light chain variable (VL) domain forms part of a therapeutic antibody. In embodiments, the heavy chain variable (VH) domain forms part of a therapeutic antibody. In embodiments, the light chain variable (VL) domain forms part of a human antibody. In embodiments, the heavy chain variable (VH) domain forms part of a human antibody. In embodiments, the light chain variable (VL) domain forms part of a humanized antibody. In embodiments, the heavy chain variable (VH) domain forms part of a humanized antibody. In embodiments, the light chain variable (VL) domain forms part of a chimeric antibody. In embodiments, the heavy chain variable (VH) domain forms part of a chimeric antibody. In embodiments, the light chain variable (VL) domain forms part of an antibody fragment. In embodiments, the heavy chain variable (VH) domain forms part of an antibody fragment. In embodiments, the light chain variable (VL) domain forms part of an antibody variant. In embodiments, the heavy chain variable (VH) domain forms part of an antibody variant. In embodiments, the light chain variable (VL) domain forms part of a Fab. In embodiments, the heavy chain variable (VH) domain forms part of a Fab. In embodiments, the light chain variable (VL) domain forms part of a scFv. In embodiments, the heavy chain variable (VH) domain forms part of a scFv.

In embodiments, the antibody is a humanized antibody. In embodiments, the antibody is a chimeric antibody. In embodiments, the antibody is a Fab′ fragment. In embodiments, the antibody is a single chain antibody (scFv).

In another aspect is provided an anti-CD3 antibody binding the same epitope as one of the CD3 antibodies provided herein including embodiments thereof. In embodiments, the epitope includes the sequence of SEQ ID NO:14 or SEQ ID NO:15. In embodiments, the epitope has the sequence of SEQ ID NO:14 or SEQ ID NO:15. In embodiments, the epitope includes the sequence of SEQ ID NO: 14. In embodiments, the epitope has the sequence of SEQ ID NO:14. In embodiments, the epitope includes the sequence of SEQ ID NO: 15. In embodiments, the epitope has the sequence of SEQ ID NO: 15.

In embodiments, the light chain variable domain includes the sequence of SEQ ID NO:7, SEQ ID NO:9, or SEQ ID NO: 11. In embodiments, the light chain variable domain includes the sequence of SEQ ID NO: 7. In embodiments, the light chain variable domain includes the sequence of SEQ ID NO: 9. In embodiments, the light chain variable domain includes the sequence of SEQ ID NO: 11. In embodiments, the light chain variable domain is the sequence of SEQ ID NO:7, SEQ ID NO: 9, or SEQ ID NO:11. In embodiments, the light chain variable domain is the sequence of SEQ ID NO: 7. In embodiments, the light chain variable domain is the sequence of SEQ ID NO:9. In embodiments, the light chain variable domain is the sequence of SEQ ID NO:11.

In embodiments, the antibody includes a light chain including the sequence of SEQ ID NO:7. In embodiments, the antibody includes a light chain with the sequence of SEQ ID NO: 7. In embodiments, the antibody includes a light chain including the sequence of SEQ ID NO:9. In embodiments, the antibody includes a light chain with the sequence of SEQ ID NO: 9. In embodiments, the antibody includes a light chain including the sequence of SEQ ID NO: 11. In embodiments, the antibody includes a light chain with the sequence of SEQ ID NO:11.

In embodiments, the antibody includes a light chain including the sequence of SEQ ID NO:29, SEQ ID NO:31, or SEQ ID NO:33. In embodiments, the antibody includes a light chain including the sequence of SEQ ID NO:29. In embodiments, the antibody includes a light chain including the sequence of SEQ ID NO:31. In embodiments, the antibody includes a light chain including the sequence of SEQ ID NO:33. In embodiments, the antibody includes a light chain with the sequence of SEQ ID NO:29, SEQ ID NO:31, or SEQ ID NO:33. In embodiments, the antibody includes a light chain with the sequence of SEQ ID NO:29. In embodiments, the antibody includes a light chain with the sequence of SEQ ID NO:31. In embodiments, the antibody includes a light chain with the sequence of SEQ ID NO:33.

In embodiments, the heavy chain variable domain includes the sequence of SEQ ID NO:8, SEQ ID NO:10 or SEQ ID NO:12. In embodiments, the heavy chain variable domain includes the sequence of SEQ ID NO:8, SEQ ID NO:10, SEQ ID NO:12, or SEQ ID NO:17. In embodiments, the heavy chain variable domain includes the sequence of SEQ ID NO: 8. In embodiments, the heavy chain variable domain includes the sequence of SEQ ID NO:10. In embodiments, the heavy chain variable domain includes the sequence of SEQ ID NO: 12. In embodiments, the heavy chain variable domain includes the sequence of SEQ ID NO: 17. In embodiments, the heavy chain variable domain is the sequence of SEQ ID NO: 8, SEQ ID NO:10, SEQ ID NO:12, or SEQ ID NO: 17. In embodiments, the heavy chain variable domain is the sequence of SEQ ID NO: 8. In embodiments, the heavy chain variable domain is the sequence of SEQ ID NO: 10. In embodiments, the heavy chain variable domain is the sequence of SEQ ID NO:12. In embodiments, the heavy chain variable domain is the sequence of SEQ ID NO:17.

In embodiments, the antibody includes a heavy chain including the sequence of SEQ ID NO:8. In embodiments, the antibody includes a heavy chain with the sequence of SEQ ID NO:8. In embodiments, the antibody includes a heavy chain including the sequence of SEQ ID NO:10. In embodiments, the antibody includes a heavy chain with the sequence of SEQ ID NO: 10. In embodiments, the antibody includes a heavy chain including the sequence of SEQ ID NO:12. In embodiments, the antibody includes a heavy chain with the sequence of SEQ ID NO:12. In embodiments, the antibody includes a heavy chain including the sequence of SEQ ID NO: 17. In embodiments, the antibody includes a heavy chain with the sequence of SEQ ID NO: 17.

In embodiments, the antibody includes a heavy chain including the sequence of SEQ ID NO:30, SEQ ID NO:32, SEQ ID NO:34, or SEQ ID NO:18. In embodiments, the antibody includes a heavy chain including the sequence of SEQ ID NO:30. In embodiments, the antibody includes a heavy chain including the sequence of SEQ ID NO:32. In embodiments, the antibody includes a heavy chain including the sequence of SEQ ID NO:34. In embodiments, the antibody includes a heavy chain including the sequence of SEQ ID NO:18. In embodiments, the antibody includes a heavy chain with the sequence of SEQ ID NO:30, SEQ ID NO:32, SEQ ID NO:34, or SEQ ID NO:18. In embodiments, the antibody includes a heavy chain with the sequence of SEQ ID NO:30. In embodiments, the antibody includes a heavy chain with the sequence of SEQ ID NO:32. In embodiments, the antibody includes a heavy chain with the sequence of SEQ ID NO:34. In embodiments, the antibody includes a heavy chain with the sequence of SEQ ID NO: 18

In embodiments, the heavy chain variable domain includes a serine at a position corresponding to position 53 of SEQ ID NO:8. In embodiments, the serine at a position corresponding to position 53 of SEQ ID NO:8 prevents oxidation at this position.

In embodiments, the light chain variable domain includes the sequence of SEQ ID NO:7 and the heavy chain variable domain includes the sequence of SEQ ID NO: 8. In embodiments, the light chain variable domain is the sequence of SEQ ID NO:7 and the heavy chain variable domain is the sequence of SEQ ID NO:8. In one further embodiment, the antibody is humanized antibody 26H8.

In embodiments, the light chain variable domain includes the sequence of SEQ ID NO:9 and the heavy chain variable domain includes the sequence of SEQ ID NO: 10. In embodiments, the light chain variable domain is the sequence of SEQ ID NO:9 and the heavy chain variable domain is the sequence of SEQ ID NO:10. In one further embodiment, the antibody is mouse antibody 26H8.

In embodiments, the light chain variable domain includes the sequence of SEQ ID NO:7 and the heavy chain variable domain includes the sequence of SEQ ID NO:17. In embodiments, the light chain variable domain is the sequence of SEQ ID NO:7 and the heavy chain variable domain is the sequence of SEQ ID NO:17. In one further embodiment, the antibody is humanized antibody 26H8 C53S.

In embodiments, the light chain variable domain includes the sequence of SEQ ID NO:11 and the heavy chain variable domain includes the sequence of SEQ ID NO: 12. In embodiments, the light chain variable domain is the sequence of SEQ ID NO: 11 and the heavy chain variable domain is the sequence of SEQ ID NO:12. In one further embodiment, the antibody is antibody 3F11.

In embodiments, the light chain includes the sequence of SEQ ID NO:29, SEQ ID NO:31, or SEQ ID NO:33; and the heavy chain includes the sequence of SEQ ID NO:30, SEQ ID NO:32, SEQ ID NO:34, or SEQ ID NO:18. In embodiments, the light chain includes the sequence of SEQ ID NO:29 and the heavy chain includes the sequence of SEQ ID NO:30. In embodiments, the light chain includes the sequence of SEQ ID NO:31 and the heavy chain includes the sequence of SEQ ID NO:32. In embodiments, the light chain includes the sequence of SEQ ID NO:33 and the heavy chain includes the sequence of SEQ ID NO:34. In embodiments, the light chain includes the sequence of SEQ ID NO:29 and the heavy chain includes the sequence of SEQ ID NO:18.

In embodiments, the light chain is the sequence of SEQ ID NO:29, SEQ ID NO:31, or SEQ ID NO:33; and the heavy chain is the sequence of SEQ ID NO:30, SEQ ID NO:32, SEQ ID NO:34, or SEQ ID NO:18. In embodiments, the light chain is the sequence of SEQ ID NO:29 and the heavy chain is the sequence of SEQ ID NO:30. In embodiments, the light chain is the sequence of SEQ ID NO:31 and the heavy chain is the sequence of SEQ ID NO:32. In embodiments, the light chain is the sequence of SEQ ID NO:33 and the heavy chain is the sequence of SEQ ID NO:34. In embodiments, the light chain is the sequence of SEQ ID NO:29 and the heavy chain is the sequence of SEQ ID NO:18.

In embodiments, the antibody is bound to a CD3 protein. In embodiments, the CD3 protein is a human CD3 protein. In embodiments, the CD3 protein is a non-human primate CD3 protein. In embodiments, the CD3 protein forms part of a cell. In embodiments, the CD3 protein is expressed on the surface of a cell. In embodiments, the cell is a T cell. In embodiments, the cell is an effector cell. In embodiments, the effector cell is a cytotoxic T cell, a helper T cell, a B cell, or a natural killer cell. Thus, in embodiments, the cell is a cytotoxic T cell. In embodiments, the cell is a helper T cell. In embodiments, the cell is a B cell. In embodiments, the cell is a natural killer cell.

In embodiments, the antibody is bound to a therapeutic moiety or a diagnostic moiety. In embodiments, the antibody is bound to a therapeutic moiety. In embodiments, the antibody is bound to a diagnostic moiety. In further embodiments, the diagnostic moiety is a detectable moiety.

Provided herein are, inter alia, non-CD3 binding antibodies that are derived from the anti-CD3 antibodies provided herein including embodiments thereof (e.g., the anti-CD3 antibody including a heavy chain variable domain of SEQ ID NO:8), and which due to the presence of an amino acid substitution (e.g., at a position corresponding to position 33 or 35) have lost their ability to bind to CD3. While the amino acid substitution does not impact the overall structural integrity of the antibody, it effectively abrogates the binding of the antibody to CD3 at a detectable level. Thus, in embodiments, the heavy chain variable domain includes a serine at a position corresponding to position 33 of SEQ ID NO:8. In embodiments, the heavy chain variable domain includes the sequence of SEQ ID NO:27. In embodiments, the heavy chain variable domain is the sequence of SEQ ID NO:27. In embodiments, the heavy chain includes the sequence of SEQ ID NO:28. In embodiments, the heavy chain is the sequence of SEQ ID NO:28.

In embodiments, the heavy chain variable domain includes a phenylalanine, a tyrosine or a glutamine at a position corresponding to position 35 of SEQ ID NO:8. In embodiments, the heavy chain variable domain includes a phenylalanine at a position corresponding to position 35 of SEQ ID NO:8. In embodiments, the heavy chain variable domain includes a tyrosine at a position corresponding to position 35 of SEQ ID NO:8. In embodiments, the heavy chain variable domain includes a glutamine at a position corresponding to position 35 of SEQ ID NO:8.

In embodiments, the heavy chain variable domain includes the sequence of SEQ ID NO:21, SEQ ID NO:23, or SEQ ID NO:25. In embodiments, the heavy chain variable domain includes the sequence of SEQ ID NO:21. In embodiments, the heavy chain variable domain includes the sequence of SEQ ID NO:23. In embodiments, the heavy chain variable domain includes the sequence of SEQ ID NO:25. In embodiments, the heavy chain variable domain is the sequence of SEQ ID NO:21, SEQ ID NO:23, or SEQ ID NO:25. In embodiments, the heavy chain variable domain is the sequence of SEQ ID NO:21. In embodiments, the heavy chain variable domain is the sequence of SEQ ID NO:23. In embodiments, the heavy chain variable domain is the sequence of SEQ ID NO:25.

In embodiments, the heavy chain includes the sequence of SEQ ID NO:22, SEQ ID NO:24, or SEQ ID NO:26. In embodiments, the heavy chain includes the sequence of SEQ ID NO:22. In embodiments, the heavy chain includes the sequence of SEQ ID NO:24. In embodiments, the heavy chain includes the sequence of SEQ ID NO:26. In embodiments, the heavy chain is the sequence of SEQ ID NO:22, SEQ ID NO:24, or SEQ ID NO:26. In embodiments, the heavy chain is the sequence of SEQ ID NO:22. In embodiments, the heavy chain is the sequence of SEQ ID NO:24. In embodiments, the heavy chain is the sequence of SEQ ID NO:26.

In embodiments, the light chain includes the sequence of SEQ ID NO:29; and the heavy chain includes the sequence of SEQ ID NO:22, SEQ ID NO:24, SEQ ID NO:26, or SEQ ID NO:28. In embodiments, the light chain includes the sequence of SEQ ID NO:29 and the heavy chain includes the sequence of SEQ ID NO:22. In embodiments, the light chain includes the sequence of SEQ ID NO:29 and the heavy chain includes the sequence of SEQ ID NO:24. In embodiments, the light chain includes the sequence of SEQ ID NO:29 and the heavy chain includes the sequence of SEQ ID NO:26. In embodiments, the light chain includes the sequence of SEQ ID NO:29 and the heavy chain includes the sequence of SEQ ID NO:28.

In embodiments, the light chain is the sequence of SEQ ID NO:29; and the heavy chain is the sequence of SEQ ID NO:22, SEQ ID NO:24, SEQ ID NO:26, or SEQ ID NO:28. In embodiments, the light chain is the sequence of SEQ ID NO:29 and the heavy chain is the sequence of SEQ ID NO:22. In embodiments, the light chain is the sequence of SEQ ID NO:29 and the heavy chain is the sequence of SEQ ID NO:24. In embodiments, the light chain is the sequence of SEQ ID NO:29 and the heavy chain is the sequence of SEQ ID NO:26. In embodiments, the light chain is the sequence of SEQ ID NO:29 and the heavy chain is the sequence of SEQ ID NO:28.

In embodiments, the antibody does not bind to CD3. In embodiments, the antibody does not bind to CD3 at a detectable level. A detectable level as provided herein is a level of binding between two proteins (the antibody provided herein and CD3) that is detected relative to a standard control using standard procedures well known in the art (e.g., immunofluorescence, Western blot, etc.).

The ability of an antibody to bind a specific epitope (e.g., human or non-human primate CD3) can be described by the equilibrium dissociation constant (K_{D}). The equilibrium dissociation constant (K_{D}) as defined herein is the ratio of the dissociation rate (K-off) and the association rate (K-on) of the anti-CD3 antibody provided herein including embodiments thereof. It is described by the following formula: K_{D}=K-off/K-on. Thus, in embodiments, the anti-CD3 antibody is capable of binding a CD3 protein with an equilibrium dissociation constant (K_{D}) of about 20 nM to about 60 nM. In embodiments, the anti-CD3 antibody is capable of binding a CD3 protein with an equilibrium dissociation constant (K_{D}) of about 25 nM to about 60 nM. In embodiments, the anti-CD3 antibody is capable of binding a CD3 protein with an equilibrium dissociation constant (K_{D}) of about 30 nM to about 60 nM. In embodiments, the anti-CD3 antibody is capable of binding a CD3 protein with an equilibrium dissociation constant (K_{D}) of about 35 nM to about 60 nM. In embodiments, the anti-CD3 antibody is capable of binding a CD3 protein with an equilibrium dissociation constant (K_{D}) of about 40 nM to about 60 nM. In embodiments, the anti-CD3 antibody is capable of binding a CD3 protein with an equilibrium dissociation constant (K_{D}) of about 45 nM to about 60 nM. In embodiments, the anti-CD3 antibody is capable of binding a CD3 protein with an equilibrium dissociation constant (K_{D}) of about 50 nM to about 60 nM. In embodiments, the anti-CD3 antibody is capable of binding a CD3 protein with an equilibrium dissociation constant (K_{D}) of about 55 nM to about 60 nM.

In embodiments, the anti-CD3 antibody is capable of binding a CD3 protein with an equilibrium dissociation constant (K_{D}) of 20 nM to 60 nM. In embodiments, the anti-CD3 antibody is capable of binding a CD3 protein with an equilibrium dissociation constant (K_{D}) of 25 nM to 60 nM. In embodiments, the anti-CD3 antibody is capable of binding a CD3 protein with an equilibrium dissociation constant (K_{D}) of 30 nM to 60 nM. In embodiments, the anti-CD3 antibody is capable of binding a CD3 protein with an equilibrium dissociation constant (K_{D}) of 35 nM to 60 nM. In embodiments, the anti-CD3 antibody is capable of binding a CD3 protein with an equilibrium dissociation constant (K_{D}) of 40 nM to 60 nM. In embodiments, the anti-CD3 antibody is capable of binding a CD3 protein with an equilibrium dissociation constant (K_{D}) of 45 nM to 60 nM. In embodiments, the anti-CD3 antibody is capable of binding a CD3 protein with an equilibrium dissociation constant (K_{D}) of 50 nM to 60 nM. In embodiments, the anti-CD3 antibody is capable of binding a CD3 protein with an equilibrium dissociation constant (K_{D}) of 55 nM to 60 nM.

In embodiments, the anti-CD3 antibody is capable of binding a CD3 protein with an equilibrium dissociation constant (K_{D}) of about 20 nM to about 55 nM. In embodiments, the anti-CD3 antibody is capable of binding a CD3 protein with an equilibrium dissociation constant (K_{D}) of about 20 nM to about 50 nM. In embodiments, the anti-CD3 antibody is capable of binding a CD3 protein with an equilibrium dissociation constant (K_{D}) of about 20 nM to about 45 nM. In embodiments, the anti-CD3 antibody is capable of binding a CD3 protein with an equilibrium dissociation constant (K_{D}) of about 20 nM to about 40 nM. In embodiments, the anti-CD3 antibody is capable of binding a CD3 protein with an equilibrium dissociation constant (K_{D}) of about 20 nM to about 35 nM. In embodiments, the anti-CD3 antibody is capable of binding a CD3 protein with an equilibrium dissociation constant (K_{D}) of about 20 nM to about 30 nM. In embodiments, the anti-CD3 antibody is capable of binding a CD3 protein with an equilibrium dissociation constant (K_{D}) of about 20 nM to about 25 nM.

In embodiments, the anti-CD3 antibody is capable of binding a CD3 protein with an equilibrium dissociation constant (K_{D}) of 20 nM to 55 nM. In embodiments, the anti-CD3 antibody is capable of binding a CD3 protein with an equilibrium dissociation constant (K_{D}) of 20 nM to 50 nM. In embodiments, the anti-CD3 antibody is capable of binding a CD3 protein with an equilibrium dissociation constant (K_{D}) of 20 nM to 45 nM. In embodiments, the anti-CD3 antibody is capable of binding a CD3 protein with an equilibrium dissociation constant (K_{D}) of 20 nM to 40 nM. In embodiments, the anti-CD3 antibody is capable of binding a CD3 protein with an equilibrium dissociation constant (K_{D}) of 20 nM to 35 nM. In embodiments, the anti-CD3 antibody is capable of binding a CD3 protein with an equilibrium dissociation constant (K_{D}) of 20 nM to 30 nM. In embodiments, the anti-CD3 antibody is capable of binding a CD3 protein with an equilibrium dissociation constant (K_{D}) of 20 nM to 25 nM. In embodiments, the anti-CD3 antibody is capable of binding a CD3 protein with an equilibrium dissociation constant (K_{D}) of about 20, 25, 30, 35, 40, 45, 50, 55, or 60 nM.

In embodiments, the anti-CD3 antibody is capable of binding a CD3 protein with an equilibrium dissociation constant (K_{D}) of about 49 nM. In embodiments, the anti-CD3 antibody is capable of binding a CD3 protein with an equilibrium dissociation constant (K_{D}) of 49 nM. In embodiments, the anti-CD3 antibody is capable of binding a CD3 protein with an equilibrium dissociation constant (K_{D}) of about 36 nM. In embodiments, the anti-CD3 antibody is capable of binding a CD3 protein with an equilibrium dissociation constant (K_{D}) of 36 nM. In embodiments, the CD3 is a human CD3 or a cynomolgus CD3. In embodiments, the anti-CD3 antibody is capable to bind a human CD3 and a cynomolgus CD3.

In embodiments, the anti-CD3 antibody is capable of binding a CD3 protein with an equilibrium dissociation constant (K_{D}) of about 25 nM to about 125 nM. In embodiments, the anti-CD3 antibody is capable of binding a CD3 protein with an equilibrium dissociation constant (K_{D}) of about 35 nM to about 125 nM. In embodiments, the anti-CD3 antibody is capable of binding a CD3 protein with an equilibrium dissociation constant (K_{D}) of about 45 nM to about 125 nM. In embodiments, the anti-CD3 antibody is capable of binding a CD3 protein with an equilibrium dissociation constant (K_{D}) of about 55 nM to about 125 nM. In embodiments, the anti-CD3 antibody is capable of binding a CD3 protein with an equilibrium dissociation constant (K_{D}) of about 65 nM to about 125 nM. In embodiments, the anti-CD3 antibody is capable of binding a CD3 protein with an equilibrium dissociation constant (K_{D}) of about 75 nM to about 125 nM. In embodiments, the anti-CD3 antibody is capable of binding a CD3 protein with an equilibrium dissociation constant (K_{D}) of about 85 nM to about 125 nM. In embodiments, the anti-CD3 antibody is capable of binding a CD3 protein with an equilibrium dissociation constant (K_{D}) of about 95 nM to about 125 nM. In embodiments, the anti-CD3 antibody is capable of binding a CD3 protein with an equilibrium dissociation constant (K_{D}) of about 105 nM to about 125 nM. In embodiments, the anti-CD3 antibody is capable of binding a CD3 protein with an equilibrium dissociation constant (K_{D}) of about 115 nM to about 125 nM.

In embodiments, the anti-CD3 antibody is capable of binding a CD3 protein with an equilibrium dissociation constant (K_{D}) of about 25 nM to about 115 nM. In embodiments, the anti-CD3 antibody is capable of binding a CD3 protein with an equilibrium dissociation constant (K_{D}) of about 25 nM to about 105 nM. In embodiments, the anti-CD3 antibody is capable of binding a CD3 protein with an equilibrium dissociation constant (K_{D}) of about 25 nM to about 95 nM. In embodiments, the anti-CD3 antibody is capable of binding a CD3 protein with an equilibrium dissociation constant (K_{D}) of about 25 nM to about 85 nM. In embodiments, the anti-CD3 antibody is capable of binding a CD3 protein with an equilibrium dissociation constant (K_{D}) of about 25 nM to about 75 nM. In embodiments, the anti-CD3 antibody is capable of binding a CD3 protein with an equilibrium dissociation constant (K_{D}) of about 25 nM to about 65 nM. In embodiments, the anti-CD3 antibody is capable of binding a CD3 protein with an equilibrium dissociation constant (K_{D}) of about 25 nM to about 55 nM. In embodiments, the anti-CD3 antibody is capable of binding a CD3 protein with an equilibrium dissociation constant (K_{D}) of about 25 nM to about 45 nM. In embodiments, the anti-CD3 antibody is capable of binding a CD3 protein with an equilibrium dissociation constant (K_{D}) of about 25 nM to about 35 nM.

In embodiments, the anti-CD3 antibody is capable of binding a CD3 protein with an equilibrium dissociation constant (K_{D}) of 25 nM to 125 nM. In embodiments, the anti-CD3 antibody is capable of binding a CD3 protein with an equilibrium dissociation constant (K_{D}) of 35 nM to 125 nM. In embodiments, the anti-CD3 antibody is capable of binding a CD3 protein with an equilibrium dissociation constant (K_{D}) of 45 nM to 125 nM. In embodiments, the anti-CD3 antibody is capable of binding a CD3 protein with an equilibrium dissociation constant (K_{D}) of 55 nM to 125 nM. In embodiments, the anti-CD3 antibody is capable of binding a CD3 protein with an equilibrium dissociation constant (K_{D}) of 65 nM to 125 nM. In embodiments, the anti-CD3 antibody is capable of binding a CD3 protein with an equilibrium dissociation constant (K_{D}) of 75 nM to 125 nM. In embodiments, the anti-CD3 antibody is capable of binding a CD3 protein with an equilibrium dissociation constant (K_{D}) of 85 nM to 125 nM. In embodiments, the anti-CD3 antibody is capable of binding a CD3 protein with an equilibrium dissociation constant (K_{D}) of 95 nM to 125 nM. In embodiments, the anti-CD3 antibody is capable of binding a CD3 protein with an equilibrium dissociation constant (K_{D}) of 105 nM to 125 nM. In embodiments, the anti-CD3 antibody is capable of binding a CD3 protein with an equilibrium dissociation constant (K_{D}) of 115 nM to 125 nM.

In embodiments, the anti-CD3 antibody is capable of binding a CD3 protein with an equilibrium dissociation constant (K_{D}) of 25 nM to 115 nM. In embodiments, the anti-CD3 antibody is capable of binding a CD3 protein with an equilibrium dissociation constant (K_{D}) of 25 nM to 105 nM. In embodiments, the anti-CD3 antibody is capable of binding a CD3 protein with an equilibrium dissociation constant (K_{D}) of 25 nM to 95 nM. In embodiments, the anti-CD3 antibody is capable of binding a CD3 protein with an equilibrium dissociation constant (K_{D}) of 25 nM to 85 nM. In embodiments, the anti-CD3 antibody is capable of binding a CD3 protein with an equilibrium dissociation constant (K_{D}) of 25 nM to 75 nM. In embodiments, the anti-CD3 antibody is capable of binding a CD3 protein with an equilibrium dissociation constant (K_{D}) of 25 nM to 65 nM. In embodiments, the anti-CD3 antibody is capable of binding a CD3 protein with an equilibrium dissociation constant (K_{D}) of 25 nM to 55 nM. In embodiments, the anti-CD3 antibody is capable of binding a CD3 protein with an equilibrium dissociation constant (K_{D}) of 25 nM to 45 nM. In embodiments, the anti-CD3 antibody is capable of binding a CD3 protein with an equilibrium dissociation constant (K_{D}) of 25 nM to 35 nM.

In embodiments, the anti-CD3 antibody is capable of binding a CD3 protein with an equilibrium dissociation constant (K_{D}) of about 51.6 nM. In embodiments, the anti-CD3 antibody is capable of binding a CD3 protein with an equilibrium dissociation constant (K_{D}) of 51.6 nM. In embodiments, the anti-CD3 antibody is capable of binding a CD3 protein with an equilibrium dissociation constant (K_{D}) of about 116 nM. In embodiments, the anti-CD3 antibody is capable of binding a CD3 protein with an equilibrium dissociation constant (K_{D}) of 116 nM. In embodiments, the CD3 is a human CD3 or a cynomolgus CD3. In embodiments, the anti-CD3 antibody is capable to bind a human CD3 and a cynomolgus CD3.

In one embodiment, the anti-CD3 antibody is a Fab′ fragment including a light chain with the sequence of SEQ ID NO:29 and a heavy chain with the sequence of SEQ ID NO:30. In one further embodiment, the anti-CD3 antibody is a Fab′ fragment referred to herein as h26H8.

In one embodiment, the anti-CD3 antibody is a Fab′ fragment including a light chain with the sequence of SEQ ID NO:31 and a heavy chain with the sequence of SEQ ID NO:32. In one further embodiment, the anti-CD3 antibody is a Fab′ fragment referred to herein as 26H8.

In one embodiment, the anti-CD3 antibody is a Fab′ fragment including a light chain with the sequence of SEQ ID NO:33 and a heavy chain with the sequence of SEQ ID NO:34. In one further embodiment, the anti-CD3 antibody is a Fab′ fragment referred to herein as 3F11.

In one embodiment, the anti-CD3 antibody is a Fab′ fragment including a light chain with the sequence of SEQ ID NO:29 and a heavy chain with the sequence of SEQ ID NO:18. In one further embodiment, the anti-CD3 antibody is a Fab′ fragment referred to herein as h26H8 C53S.

In one embodiment, the anti-CD3 antibody is a Fab′ fragment including a light chain with the sequence of SEQ ID NO:29 and a heavy chain with the sequence of SEQ ID NO:22. In one further embodiment, the anti-CD3 antibody is a Fab′ fragment referred to herein as h26H8 H35F.

In one embodiment, the anti-CD3 antibody is a Fab′ fragment including a light chain with the sequence of SEQ ID NO:29 and a heavy chain with the sequence of SEQ ID NO:24. In one further embodiment, the anti-CD3 antibody is a Fab′ fragment referred to herein as h26H8 H35Y.

In one embodiment, the anti-CD3 antibody is a Fab′ fragment including a light chain with the sequence of SEQ ID NO:29 and a heavy chain with the sequence of SEQ ID NO:26. In one further embodiment, the anti-CD3 antibody is a Fab′ fragment referred to herein as h26H8 H35Q.

In one embodiment, the anti-CD3 antibody is a Fab′ fragment including a light chain with the sequence of SEQ ID NO:29 and a heavy chain with the sequence of SEQ ID NO:28. In one further embodiment, the anti-CD3 antibody is a Fab′ fragment referred to herein as h26H8 Y33S.

### Recombinant Protein Compositions

As described above, the light chain variable (VL) domain and the heavy chain variable (VH) domain provided herein including embodiments thereof, may each independently form part of an antibody, an antibody variant, a fragment of an antibody, a fragment of an antibody variant, or a recombinant protein (e.g., a chimeric antigen receptor (CAR) or a bispecific antibody). Provided herein are, inter alia, recombinant proteins (e.g., a CAR or a bispecific antibody), which include the light chain variable (VL) domain and/or the heavy chain variable (VH) domain as provided herein and are therefore capable of binding CD3 on T cells as well as a target cell ligand on, for example, a cancer cell. In embodiments, the recombinant protein is a bispecific antibody.

An “antibody region” as provided herein refers to a monovalent or multivalent protein moiety that forms part of the recombinant protein (e.g., CAR or bispecific antibody) provided herein including embodiments thereof. A person of ordinary skill in the art will therefore immediately recognize that the antibody region is a protein moiety capable of binding an antigen (epitope). Thus, the antibody region provided herein may include a domain of an antibody (e.g., a light chain variable (VL) domain, a heavy chain variable (VH) domain) or a fragment of an antibody (e.g., Fab). In embodiments, the antibody region is a protein conjugate. A “protein conjugate” as provided herein refers to a construct consisting of more than one polypeptide, wherein the polypeptides are bound together covalently or non-covalently. In embodiments, the protein conjugate includes a Fab moiety (a monovalent Fab) covalently attached to an scFv moiety (a monovalent scFv). In embodiments, the protein conjugate includes a plurality (at least two) Fab moieties. In embodiments, the polypeptides of a protein conjugate are encoded by one nucleic acid molecule. In embodiments, the polypeptides of a protein conjugate are encoded by different nucleic acid molecules. In embodiments, the polypeptides are connected through a linker. In embodiments, the polypeptides are connected through a chemical linker. In embodiments, the antibody region is an scFv. The antibody region may include a light chain variable (VL) domain and/or a heavy chain variable (VH) domain. Thus, in embodiments, the antibody region includes a single domain antibody. In embodiments, the antibody region includes a light chain variable (VL) domain. In embodiments, the antibody region includes a heavy chain variable (VH) domain. In embodiments, the antibody region is a single domain antibody. In embodiments, the single domain antibody includes a heavy chain variable (VH) domain. In embodiments, the single domain antibody includes a light chain variable (VL) domain. In embodiments, the single domain antibody is a heavy chain variable (VH) domain. In embodiments, the single domain antibody is a light chain variable (VL) domain.

In one embodiment, the recombinant protein includes a light chain variable domain with the sequence of SEQ ID NO:7 and a heavy chain variable domain with the sequence of SEQ ID NO:8. In one further embodiment, the anti-CD3 recombinant protein is referred to herein as h26H8.

In one embodiment, the anti-CD3 recombinant protein includes a light chain variable domain with the sequence of SEQ ID NO:9 and a heavy chain variable domain with the sequence of SEQ ID NO: 10. In one further embodiment, the anti-CD3 recombinant protein is referred to herein as 26H8.

In one embodiment, the anti-CD3 recombinant protein includes a light chain variable domain with the sequence of SEQ ID NO:11 and a heavy chain variable domain with the sequence of SEQ ID NO: 12. In one further embodiment, the anti-CD3 recombinant protein is referred to herein as 3F11.

In one embodiment, the anti-CD3 recombinant protein includes a light chain variable domain the with sequence of SEQ ID NO:7 and a heavy chain variable domain with the sequence of SEQ ID NO: 17. In one further embodiment, the anti-CD3 recombinant protein is referred to herein as h26H8 C53S.

In one embodiment, the anti-CD3 recombinant protein includes a light chain variable domain the with sequence of SEQ ID NO:7 and a heavy chain variable domain with the sequence of SEQ ID NO:21. In one further embodiment, the anti-CD3 recombinant protein is referred to herein as h26H8 H35F.

In one embodiment, the anti-CD3 recombinant protein includes a light chain variable domain the with sequence of SEQ ID NO:7 and a heavy chain variable domain with the sequence of SEQ ID NO:23. In one further embodiment, the anti-CD3 recombinant protein is referred to herein as h26H8 H35Y.

In one embodiment, the anti-CD3 recombinant protein includes a light chain variable domain the with sequence of SEQ ID NO:7 and a heavy chain variable domain with the sequence of SEQ ID NO:25. In one further embodiment, the anti-CD3 recombinant protein is referred to herein as h26H8 H35Q.

In one embodiment, the anti-CD3 recombinant protein includes a light chain variable domain the with sequence of SEQ ID NO:7 and a heavy chain variable domain with the sequence of SEQ ID NO:27. In one further embodiment, the anti-CD3 recombinant protein is referred to herein as h26H8 Y33S.

### Bispecific Antibodies

The recombinant proteins provided herein may, inter alia, be bispecifc antibodies. Thus, the second antibody region may include any of the light chain and/or heavy chain variable domains provided herein including embodiments thereof. The light chain variable (VL) domain and/or the heavy chain variable (VH) domain as provided herein may form part of a bispecific antibody.

Thus, in another aspect is provided a recombinant protein including (i) a first antibody region capable of binding a target cell ligand; and (ii) a second antibody region, including: (a) a second light chain variable domain including a CDR L1 as set forth in SEQ ID NO: 1, a CDR L2 as set forth in SEQ ID NO:2 and a CDR L3 as set forth in SEQ ID NO:3; and (b) a second heavy chain variable domain including a CDR H1 as set forth in SEQ ID NO:4, a CDR H2 as set forth in SEQ ID NO:5, and a CDR H3 as set forth in SEQ ID NO:6.

In another aspect is provided a recombinant protein including (i) a first antibody region capable of binding a target cell ligand; and (ii) a second antibody region, including: (a) a second light chain variable domain including a CDR L1 as set forth in SEQ ID NO:1, a CDR L2 as set forth in SEQ ID NO:2 and a CDR L3 as set forth in SEQ ID NO: 3; and (b) a second heavy chain variable domain including a CDR H1 as set forth in SEQ ID NO:4, a CDR H2 as set forth in SEQ ID NO:16, and a CDR H3 as set forth in SEQ ID NO: 6.

In another aspect is provided a recombinant protein including (i) a first antibody region capable of binding a target cell ligand; and (ii) a second antibody region, including: (a) a second light chain variable domain including a CDR L1 as set forth in SEQ ID NO:1, a CDR L2 as set forth in SEQ ID NO:2 and a CDR L3 as set forth in SEQ ID NO:3; and (b) a second heavy chain variable domain including a CDR H1 as set forth in SEQ ID NO:4, a CDR H2 as set forth in SEQ ID NO:19, and a CDR H3 as set forth in SEQ ID NO:20.

In embodiments, the second light chain variable domain includes the sequence of SEQ ID NO:7, SEQ ID NO:9, or SEQ ID NO:11. In embodiments, the second light chain variable domain includes the sequence of SEQ ID NO:7. In embodiments, the second light chain variable domain includes the sequence of SEQ ID NO:9. In embodiments, the second light chain variable domain includes the sequence of SEQ ID NO: 11. In embodiments, the second light chain variable domain is the sequence of SEQ ID NO: 7, SEQ ID NO:9, or SEQ ID NO:11 In embodiments, the second light chain variable domain is the sequence of SEQ ID NO:7. In embodiments, the second light chain variable domain is the sequence of SEQ ID NO:9. In embodiments, the second light chain variable domain is the sequence of SEQ ID NO:11.

In embodiments, the recombinant protein includes a second light chain including the sequence of SEQ ID NO:7. In embodiments, the recombinant protein includes a second light chain with the sequence of SEQ ID NO:7. In embodiments, the recombinant protein includes a second light chain including the sequence of SEQ ID NO:9. In embodiments, the recombinant protein includes a second light chain with the sequence of SEQ ID NO:9. In embodiments, the recombinant protein includes a second light chain including the sequence of SEQ ID NO:11. In embodiments, the recombinant protein includes a second light chain with the sequence of SEQ ID NO:11.

In embodiments, the recombinant protein includes a second light chain including the sequence of SEQ ID NO:29, SEQ ID NO:31, or SEQ ID NO:33. In embodiments, the recombinant protein includes a second light chain including the sequence of SEQ ID NO:29. In embodiments, the recombinant protein includes a second light chain including the sequence of SEQ ID NO:31. In embodiments, the recombinant protein includes a second light chain including the sequence of SEQ ID NO:33. In embodiments, the recombinant protein includes a second light chain with the sequence of SEQ ID NO:29, SEQ ID NO:31, or SEQ ID NO:33. In embodiments, the recombinant protein includes a second light chain with the sequence of SEQ ID NO:29. In embodiments, the recombinant protein includes a second light chain with the sequence of SEQ ID NO:31. In embodiments, the recombinant protein includes a second light chain with the sequence of SEQ ID NO:33.

In embodiments, the second heavy chain variable domain includes the sequence of SEQ ID NO:8, SEQ ID NO:10 or SEQ ID NO:12. In embodiments, the second heavy chain variable domain includes the sequence of SEQ ID NO:8, SEQ ID NO:10, SEQ ID NO: 12, or SEQ ID NO:17. In embodiments, the second heavy chain variable domain includes the sequence of SEQ ID NO:8. In embodiments, the second heavy chain variable domain includes the sequence of SEQ ID NO:10. In embodiments, the second heavy chain variable domain includes the sequence of SEQ ID NO: 12. In embodiments, the second heavy chain variable domain includes the sequence of SEQ ID NO: 17. In embodiments, the second heavy chain variable domain is the sequence of SEQ ID NO:8, SEQ ID NO:10, SEQ ID NO:12, or SEQ ID NO:17. In embodiments, the second heavy chain variable domain is the sequence of SEQ ID NO:8. In embodiments, the second heavy chain variable domain is the sequence of SEQ ID NO:10. In embodiments, the second heavy chain variable domain is the sequence of SEQ ID NO:12. In embodiments, the second heavy chain variable domain is the sequence of SEQ ID NO:17.

In embodiments, the recombinant protein includes a second heavy chain including the sequence of SEQ ID NO:8. In embodiments, the recombinant protein includes a second heavy chain with the sequence of SEQ ID NO:8. In embodiments, the recombinant protein includes a second heavy chain including the sequence of SEQ ID NO:10. In embodiments, the recombinant protein includes a second heavy chain with the sequence of SEQ ID NO:10. In embodiments, the recombinant protein includes a second heavy chain including the sequence of SEQ ID NO:12. In embodiments, the recombinant protein includes a second heavy chain with the sequence of SEQ ID NO:12. In embodiments, the recombinant protein includes a second heavy chain including the sequence of SEQ ID NO:17. In embodiments, the recombinant protein includes a second heavy chain with the sequence of SEQ ID NO:17.

In embodiments, the recombinant protein includes a second heavy chain including the sequence of SEQ ID NO:30, SEQ ID NO:32, SEQ ID NO:34, or SEQ ID NO:18. In embodiments, the recombinant protein includes a second heavy chain including the sequence of SEQ ID NO:30. In embodiments, the recombinant protein includes a second heavy chain including the sequence of SEQ ID NO:32. In embodiments, the recombinant protein includes a second heavy chain including the sequence of SEQ ID NO:34. In embodiments, the recombinant protein includes a second heavy chain including the sequence of SEQ ID NO:18. In embodiments, the recombinant protein includes a second heavy chain with the sequence of SEQ ID NO:30, SEQ ID NO:32, SEQ ID NO:34, or SEQ ID NO:18. In embodiments, the recombinant protein includes a second heavy chain with the sequence of SEQ ID NO:30. In embodiments, the recombinant protein includes a second heavy chain with the sequence of SEQ ID NO:32. In embodiments, the recombinant protein includes a second heavy chain with the sequence of SEQ ID NO:34. In embodiments, the recombinant protein includes a second heavy chain with the sequence of SEQ ID NO:18.

In embodiments, the second heavy chain variable domain includes a serine at a position corresponding to position 53 of SEQ ID NO:8. In embodiments, the serine at a position corresponding to position 53 of SEQ ID NO:8 prevents oxidation at this position.

In embodiments, the second light chain variable domain includes the sequence of SEQ ID NO:7, SEQ ID NO:9, or SEQ ID NO:11; and the second heavy chain variable domain includes the sequence of SEQ ID NO:8, SEQ ID NO:10, SEQ ID NO:12, or SEQ ID NO:17. In embodiments, the second light chain variable domain includes the sequence of SEQ ID NO:7 and the second heavy chain variable domain includes the sequence of SEQ ID NO:8. In embodiments, the second light chain variable domain includes the sequence of SEQ ID NO:9 and the second heavy chain variable domain includes the sequence of SEQ ID NO:10. In embodiments, the second light chain variable domain includes the sequence of SEQ ID NO:11 and the second heavy chain variable domain includes the sequence of SEQ ID NO:12. In embodiments, the second light chain variable domain includes the sequence of SEQ ID NO:7 and the second heavy chain variable domain includes the sequence of SEQ ID NO:17.

In embodiments, the second light chain variable domain is the sequence of SEQ ID NO:7, SEQ ID NO:9, or SEQ ID NO: 11; and the second heavy chain variable domain is the sequence of SEQ ID NO:8, SEQ ID NO:10, SEQ ID NO:12, or SEQ ID NO: 17. In embodiments, the second light chain variable domain is the sequence of SEQ ID NO:7 and the second heavy chain variable domain is the sequence of SEQ ID NO:8. In embodiments, the second light chain variable domain is the sequence of SEQ ID NO:9 and the second heavy chain variable domain is the sequence of SEQ ID NO:10. In embodiments, the second light chain variable domain is the sequence of SEQ ID NO:11 and the second heavy chain variable domain is the sequence of SEQ ID NO:12. In embodiments, the second light chain variable domain is the sequence of SEQ ID NO:7 and the second heavy chain variable domain is the sequence of SEQ ID NO:17.

In embodiments, the second light chain includes the sequence of SEQ ID NO:29, SEQ ID NO:31, or SEQ ID NO:33; and the second heavy chain includes the sequence of SEQ ID NO:30, SEQ ID NO:32, SEQ ID NO:34, or SEQ ID NO:18. In embodiments, the second light chain includes the sequence of SEQ ID NO:29 and the second heavy chain includes the sequence of SEQ ID NO:30. In embodiments, the second light chain includes the sequence of SEQ ID NO:31 and the second heavy chain includes the sequence of SEQ ID NO:32. In embodiments, the second light chain includes the sequence of SEQ ID NO:33 and the second heavy chain includes the sequence of SEQ ID NO:34. In embodiments, the second light chain includes the sequence of SEQ ID NO:29 and the second heavy chain includes the sequence of SEQ ID NO:18.

In embodiments, the second light chain is the sequence of SEQ ID NO:29, SEQ ID NO:31, or SEQ ID NO:33; and the second heavy chain is the sequence of SEQ ID NO:30, SEQ ID NO:32, SEQ ID NO:34, or SEQ ID NO:18. In embodiments, the second light chain is the sequence of SEQ ID NO:29 and the second heavy chain is the sequence of SEQ ID NO:30. In embodiments, the second light chain is the sequence of SEQ ID NO:31 and the second heavy chain is the sequence of SEQ ID NO:32. In embodiments, the second light chain is the sequence of SEQ ID NO:33 and the second heavy chain is the sequence of SEQ ID NO:34. In embodiments, the second light chain is the sequence of SEQ ID NO:29 and the second heavy chain is the sequence of SEQ ID NO:18.

In embodiments, the second light chain variable domain includes the sequence of SEQ ID NO:7 and the second heavy chain variable domain includes the sequence of SEQ ID NO:8. In embodiments, the second light chain variable domain is the sequence of SEQ ID NO:7 and the second heavy chain variable domain is the sequence of SEQ ID NO:8. In one further embodiment, the second antibody region is humanized antibody 26H8.

In embodiments, the second light chain variable domain includes the sequence of SEQ ID NO:9 and the second heavy chain variable domain includes the sequence of SEQ ID NO:10. In embodiments, the second light chain variable domain is the sequence of SEQ ID NO:9 and the second heavy chain variable domain is the sequence of SEQ ID NO:10. In one further embodiment, the second antibody region is mouse antibody 26H8.

In embodiments, the second light chain variable domain includes the sequence of SEQ ID NO:11 and the second heavy chain variable domain includes the sequence of SEQ ID NO:12. In embodiments, the second light chain variable domain is the sequence of SEQ ID NO: 11 and the second heavy chain variable domain is the sequence of SEQ ID NO: 12. In one further embodiment, the second antibody region is antibody 3F11.

In embodiments, the second light chain variable domain includes the sequence of SEQ ID NO:7 and the second heavy chain variable domain includes the sequence of SEQ ID NO:17. In embodiments, the second light chain variable domain is the sequence of SEQ ID NO:7 and the second heavy chain variable domain is the sequence of SEQ ID NO:17. In one further embodiment, the second antibody region is humanized antibody 26H8 C53S.

The term “target cell ligand” as provided herein refers to a cell surface molecule expressed on a target cell. The target cell may be a cancer cell, an infected cell or any other type of cell that requires targeting and subsequent elimination by an effector cell (e.g, T cell, Nk cell). Upon binding of the second antibody region to CD3 expressed on the effector cell, the effector cell is activated and able to exert its function (e.g., selective killing or eradication of the malignant, infected or otherwise unhealthy target cell). The first antibody region and the second antibody region as provided herein may independently be an antibody, an antibody variant, a fragment of an antibody or a fragment of an antibody variant.

In embodiments, the first antibody region is bound to the target cell ligand. In embodiments, the target cell ligand is a cancer protein or fragment thereof.

In embodiments, the first antibody region is a first Fab′ fragment or the second antibody region is a second Fab′ fragment. In embodiments, the first antibody region is a first Fab′ fragment and the second antibody region is a second Fab′ fragment.

In embodiments, the first antibody region is a single chain variable fragment (scFv) or the second antibody region is a second single chain variable fragment (scFv). In embodiments, the first antibody region is a single chain variable fragment (scFv) and the second antibody region is a second single chain variable fragment (scFv). In embodiments, the first antibody region is a single chain variable fragment (scFv). In embodiments, the second antibody region is a second single chain variable fragment (scFv).

In embodiments, the target cell ligand forms part of a cell. In embodiments, the cell is a cancer cell. In embodiments, the second antibody region is bound to a CD3 protein. In embodiments, the CD3 protein is a human CD3 protein. In embodiments, the CD3 protein is a non-human primate CD3 protein. In embodiments, the CD3 protein forms part of a cell. In embodiments, the CD3 protein is expressed on the surface of a cell. In embodiments, the cell is a T cell.

Provided herein are, inter alia, non-CD3 binding recombinant proteins (proteins that do not bind CD3 at a detectable level). The non-CD3 binding recombinant proteins may include a second antibody region derived from the non-CD3 binding antibodies described herein. Thus, the non-CD3 binding recombinant proteins provided herein may include a second antibody region including an amino acid substitution (e.g., at a position corresponding to position 33 or 35) which if present in the second antibody region results in the lack of binding of the recombinant protein to CD3. While, the amino acid substitution does not impact the overall structural integrity of the second antibody region or the recombinant protein, it abrogates the binding of the recombinant protein to CD3 at a detectable level. Therefore, in embodiments the recombinant protein includes a first antibody region capable of binding a target cell ligand and a second antibody region that does not bind CD3. In embodiments, the heavy chain variable domain includes a serine at a position corresponding to position 33 of SEQ ID NO:8. In embodiments, the heavy chain variable domain includes the sequence of SEQ ID NO:27. In embodiments, the heavy chain variable domain is the sequence of SEQ ID NO:27. In embodiments, the heavy chain includes the sequence of SEQ ID NO:28. In embodiments, the heavy chain is the sequence of SEQ ID NO:28.

In embodiments, the second heavy chain variable domain includes a phenylalanine, a tyrosine or a glutamine at a position corresponding to position 35 of SEQ ID NO:8. In embodiments, the second heavy chain variable domain includes a phenylalanine at a position corresponding to position 35 of SEQ ID NO:8. In embodiments, the second heavy chain variable domain includes a tyrosine at a position corresponding to position 35 of SEQ ID NO:8. In embodiments, the second heavy chain variable domain includes a glutamine at a position corresponding to position 35 of SEQ ID NO:8.

In embodiments, the second heavy chain variable domain includes the sequence of SEQ ID NO:21, SEQ ID NO:23, or SEQ ID NO:25. In embodiments, the second heavy chain variable domain includes the sequence of SEQ ID NO:21. In embodiments, the second heavy chain variable domain includes the sequence of SEQ ID NO:23. In embodiments, the second heavy chain variable domain includes the sequence of SEQ ID NO:25. In embodiments, the second heavy chain variable domain is the sequence of SEQ ID NO:21, SEQ ID NO:23, or SEQ ID NO:25. In embodiments, the second heavy chain variable domain is the sequence of SEQ ID NO:21. In embodiments, the second heavy chain variable domain is the sequence of SEQ ID NO:23. In embodiments, the second heavy chain variable domain is the sequence of SEQ ID NO:25.

In embodiments, the second light chain variable domain includes the sequence of SEQ ID NO:7; and the second heavy chain variable domain includes the sequence of SEQ ID NO:21, SEQ ID NO:23, SEQ ID NO:25, or SEQ ID NO:27. In embodiments, the second light chain variable domain includes the sequence of SEQ ID NO:7 and the second heavy chain variable domain includes the sequence of SEQ ID NO:21. In embodiments, the second light chain variable domain includes the sequence of SEQ ID NO:7 and the second heavy chain variable domain includes the sequence of SEQ ID NO:23. In embodiments, the second light chain variable domain includes the sequence of SEQ ID NO:7 and the second heavy chain variable domain includes the sequence of SEQ ID NO:25. In embodiments, the second light chain variable domain includes the sequence of SEQ ID NO:7 and the second heavy chain variable domain includes the sequence of SEQ ID NO:27.

In embodiments, the second light chain variable domain is the sequence of SEQ ID NO: 7; and the second heavy chain variable domain is the sequence of SEQ ID NO:21, SEQ ID NO:23, SEQ ID NO:25, or SEQ ID NO:27. In embodiments, the second light chain variable domain is the sequence of SEQ ID NO:7 and the second heavy chain variable domain is the sequence of SEQ ID NO:21. In embodiments, the second light chain variable domain is the sequence of SEQ ID NO:7 and the second heavy chain variable domain is the sequence of SEQ ID NO:23. In embodiments, the second light chain variable domain is the sequence of SEQ ID NO:7 and the second heavy chain variable domain is the sequence of SEQ ID NO:25. In embodiments, the second light chain variable domain is the sequence of SEQ ID NO:7 and the second heavy chain variable domain is the sequence of SEQ ID NO:27.

In embodiments, the second heavy chain includes the sequence of SEQ ID NO:22, SEQ ID NO:24, or SEQ ID NO:26. In embodiments, the second heavy chain includes the sequence of SEQ ID NO:22. In embodiments, the second heavy chain includes the sequence of SEQ ID NO:24. In embodiments, the second heavy chain includes the sequence of SEQ ID NO:26. In embodiments, the second heavy chain is the sequence of SEQ ID NO:22, SEQ ID NO:24, or SEQ ID NO:26. In embodiments, the second heavy chain is the sequence of SEQ ID NO:22. In embodiments, the second heavy chain is the sequence of SEQ ID NO:24. In embodiments, the second heavy chain is the sequence of SEQ ID NO:26.

In embodiments, the second light chain includes the sequence of SEQ ID NO: 29; and the second heavy chain includes the sequence of SEQ ID NO:22, SEQ ID NO:24, SEQ ID NO:26, or SEQ ID NO:28. In embodiments, the second light chain includes the sequence of SEQ ID NO:29 and the second heavy chain includes the sequence of SEQ ID NO:22. In embodiments, the second light chain includes the sequence of SEQ ID NO:29 and the second heavy chain includes the sequence of SEQ ID NO:24. In embodiments, the second light chain includes the sequence of SEQ ID NO:29 and the second heavy chain includes the sequence of SEQ ID NO:26. In embodiments, the second light chain includes the sequence of SEQ ID NO:29 and the second heavy chain includes the sequence of SEQ ID NO:28.

In embodiments, the second light chain is the sequence of SEQ ID NO:29; and the second heavy chain is the sequence of SEQ ID NO:22, SEQ ID NO:24, SEQ ID NO:26, or SEQ ID NO:28. In embodiments, the second light chain is the sequence of SEQ ID NO:29 and the second heavy chain is the sequence of SEQ ID NO:22. In embodiments, the second light chain is the sequence of SEQ ID NO:29 and the second heavy chain is the sequence of SEQ ID NO:24. In embodiments, the second light chain is the sequence of SEQ ID NO:29 and the second heavy chain is the sequence of SEQ ID NO:26. In embodiments, the second light chain is the sequence of SEQ ID NO:29 and the second heavy chain is the sequence of SEQ ID NO:28.

In embodiments, the second antibody region does not bind to CD3. In embodiments, the second antibody region does not bind to CD3 at a detectable level. A detectable level as provided herein is a level of binding between two proteins (the recombinant protein provided herein and CD3) that is detected relative to a standard control using standard procedures well known in the art (e.g., immunofluorescence, Western blot, etc.).

In embodiments, the recombinant protein is bound to a therapeutic moiety or a diagnostic moiety. In embodiments, the recombinant protein is bound to a therapeutic moiety. In embodiments, the recombinant protein is bound to a diagnostic moiety. In further embodiments, the diagnostic moiety is a detectable moiety.

In one embodiment, the anti-CD3 recombinant protein includes a second light chain with the sequence of SEQ ID NO:29 and a second heavy chain with the sequence of SEQ ID NO:30. In one further embodiment, the anti-CD3 recombinant protein is referred to herein as h26H8.

In one embodiment, the anti-CD3 recombinant protein includes a second light chain with the sequence of SEQ ID NO:31 and a second heavy chain with the sequence of SEQ ID NO:32. In one further embodiment, the anti-CD3 recombinant protein is referred to herein as 26H8.

In one embodiment, the anti-CD3 recombinant protein includes a second light chain with the sequence of SEQ ID NO:33 and a second heavy chain with the sequence of SEQ ID NO:34. In one further embodiment, the anti-CD3 recombinant protein is referred to herein as 3F11.

In one embodiment, the anti-CD3 recombinant protein includes a second light chain with the sequence of SEQ ID NO:29 and a second heavy chain with the sequence of SEQ ID NO:18. In one further embodiment, the anti-CD3 recombinant protein is referred to herein as h26H8 C53S.

In one embodiment, the anti-CD3 recombinant protein includes a second light chain with the sequence of SEQ ID NO:29 and a second heavy chain with the sequence of SEQ ID NO:22. In one further embodiment, the anti-CD3 recombinant protein is referred to herein as h26H8 H35F.

In one embodiment, the anti-CD3 recombinant protein includes a second light chain with the sequence of SEQ ID NO:29 and a second heavy chain with the sequence of SEQ ID NO:24. In one further embodiment, the anti-CD3 recombinant protein is referred to herein as h26H8 H35Y.

In one embodiment, the anti-CD3 recombinant protein includes a second light chain with the sequence of SEQ ID NO:29 and a second heavy chain with the sequence of SEQ ID NO:26. In one further embodiment, the anti-CD3 recombinant protein is referred to herein as h26H8 H35Q.

In one embodiment, the anti-CD3 recombinant protein includes a second light chain with the sequence of SEQ ID NO:29 and a second heavy chain with the sequence of SEQ ID NO:28. In one further embodiment, the anti-CD3 recombinant protein is referred to herein as h26H8 Y33S.

### Pharmaceutical Compositions

The antibodies and recombinant proteins provided herein including embodiments thereof may form part of a pharmaceutical compositions. Thus, in another aspect is provided a pharmaceutical composition including (i) a therapeutically effective amount of an anti-CD3 antibody as provided herein including embodiments thereof, or a therapeutically effective amount of a recombinant protein as provided herein including embodiments thereof and (ii) a pharmaceutically acceptable excipient.

### Methods of Treatment

The compositions (e.g., the anti CD3 antibodies and recombinant proteins) provided herein, including embodiments thereof, are contemplated as providing effective treatments for diseases such as cancer (e.g., triple negative breast cancer, lung cancer, pancreatic cancer). Thus, in an aspect is provided a method of treating cancer in a subject in need thereof, the method including administering to a subject a therapeutically effective amount of an anti-CD3 antibody as provided herein including embodiments thereof or a therapeutically effective amount of a recombinant protein as provided herein including embodiments thereof, thereby treating cancer in the subject.

In an aspect is provided a method of treating cancer in a subject in need thereof, the method including administering to a subject a therapeutically effective amount of an anti-CD3 antibody as provided herein including embodiments thereof. In embodiments, the antibody is attached to a therapeutic moiety.

In an aspect is provided a method of treating cancer in a subject in need thereof, the method including administering to a subject a therapeutically effective amount of a recombinant protein as provided herein including embodiments thereof, thereby treating cancer in the subject.

In an aspect is provided a method of treating a disease in a subject in need thereof, the method including administering to a subject a therapeutically effective amount of an anti-CD3 antibody as provided herein including embodiments thereof, or a therapeutically effective amount of a recombinant protein as provided herein including embodiments thereof, thereby treating the disease in the subject. In embodiments, the disease is cancer.

### Nucleic Acid Compositions

The compositions provided herein include nucleic acid molecules encoding the anti-CD3 antibodies and recombinant proteins provided herein including embodiments thereof. Thus, in an aspect is provided an isolated nucleic acid encoding an anti-CD3 antibody as provided herein including embodiments thereof.

In another aspect is provided a cell including an anti-CD3 antibody as provided herein including embodiments thereof.

In another aspect is provided a cell including a recombinant protein as provided herein including embodiments thereof.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Methods of Forming Antibodies

In an aspect provided is a method of forming an anti-CD3 antibody, the method including administering a polypeptide including the sequence of SEQ ID NO:14 or SEQ ID NO:15 to a mammal, thereby forming an anti-CD3 antibody. In embodiments, the mammal is a mouse. In embodiments, the method includes administering an adjuvant to the mammal.

### Examples

The present invention relates to monoclonal antibodies binding to human and monkey CD3 antigens and uses of such antibodies. Specifically, the antibodies recognize share N-terminal sequences of human and monkey CD3$ chains and stimulate T cells producing cytokines upon antibody binding. Encoded amino acid sequences of heavy and light chains are provided.

### Production of Recombinant CD3 Proteins

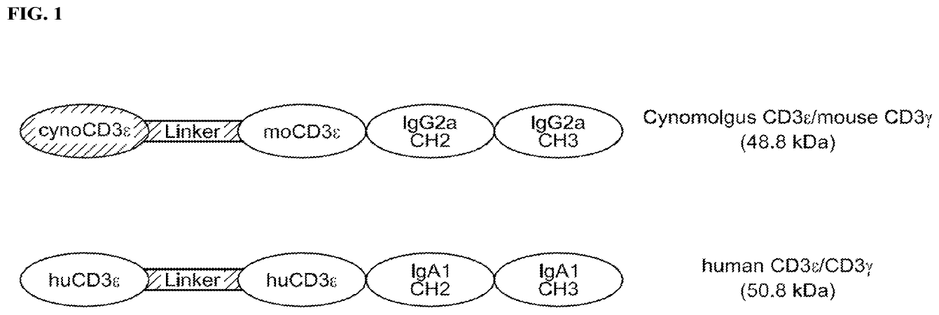

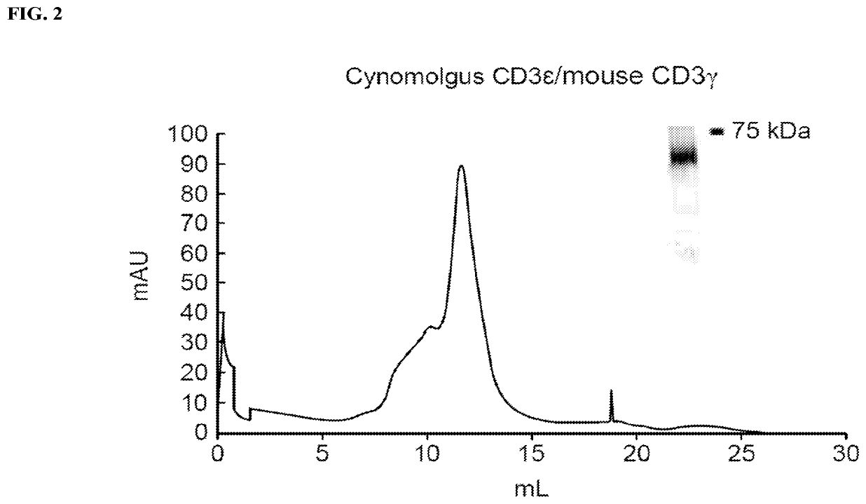

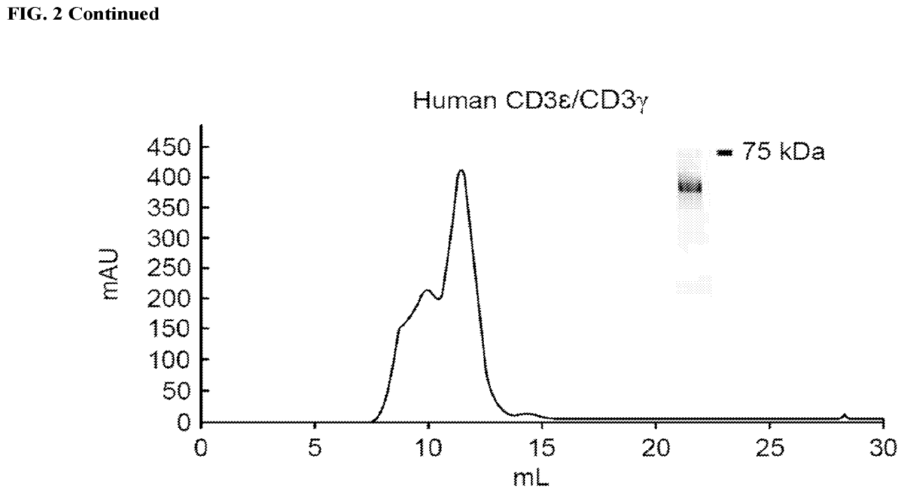

To construct recombinant CD3 fusion proteins, the extracellular domain of CD3 was fused with mouse IgG2a.Fc or human IgA1.Fc, respectively (FIG. 1, seq. 01-02). Recombinant DNAs encoding these two proteins were synthesized and cloned into a mammalian expression vector by the manufacture (Twist Bioscience). The CD3 proteins were produced using an ExpiCHO expression system (Thermo Fisher Scientific). The procedures were followed according to the manufacturer's manual. In brief, CHO cells were seeded at 3-4×106 cells/mL in fresh medium one day before transfection. On the next day cells were adjusted to 6×106 cells/mL with fresh medium. For a 100-mL transfection, 80 μg of DNA was added into 4 mL of OptiPRO™ SFM and then mixed with 320 μL of ExpiFectamine™ diluted in 3.7 mL OptiPRO™ SFM. The mixture was slowly added into the cell culture with gentle swirling, and cells were then cultured at 37° C. for 16-22 h. ExpiCHO™ Enhancer (0.6 mL) and ExpiCHO™ Feed (24 mL) were added into the cell culture, and then cells were cultured at 32° C. for 10 days. Culture supernatants were harvested by centrifugation at 4000×g for 30 minutes and passed through 0.22-μm filters for protein purification. Protein A resins (GE Healthcare) and CaptureSelect™ IgA Affinity Matrix (Thermo Fisher Scientific) were used to purify recombinant cynomolgus CD3ε/mouse CD3γ and human CD3ε/CD3γ proteins, respectively. Purification procedures were followed by the manufacturer's manuals. Purified proteins were revealed by using reduced SDS-PAGE with Mini-PROTEAN® TGX Stain-Free™ Precast Gels (Bio-Rad) and a Superdex 200 Increase 10/300 GL column (GE Healthcare) run with PBS (FIG. 2).

### Preparations of Cross-Reactive Anti-CD3 Hybridomas

All animal experiments were conducted under the approval of Institutional Animal Care and Use Committee of City of Hope (IUCAC #19070). For mouse immunization, recombinant cynomolgus CD3ε/mouse CD3γ proteins emulsified with complete Freund's adjuvants (Sigma Aldrich) were subcutaneously injected into 10 Balb/c mice (The Jackson Laboratory). Fifty micrograms of proteins were injected for each mouse. After three weeks, mice received two subcutaneous injections with 50 μg of CD3 proteins emulsified with incomplete Freund's adjuvants (Sigma Aldrich) in a two-week interval. Three days before spleen harvests, 10 μg of CD3 proteins were injected into mice via tail veins. Spleen cells were harvested for fusion with mouse myeloma cell line FO (ATCC) using PEG 1500 (Roche). The cell fusion procedures were followed according to the manufacturer's manual.

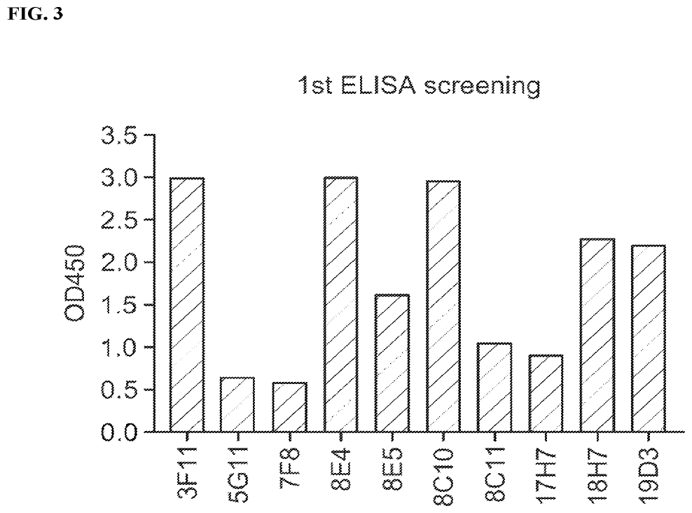

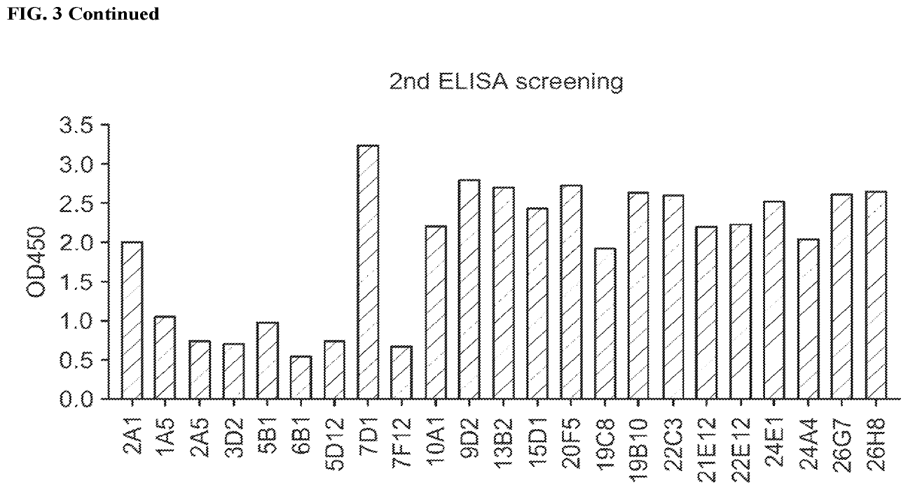

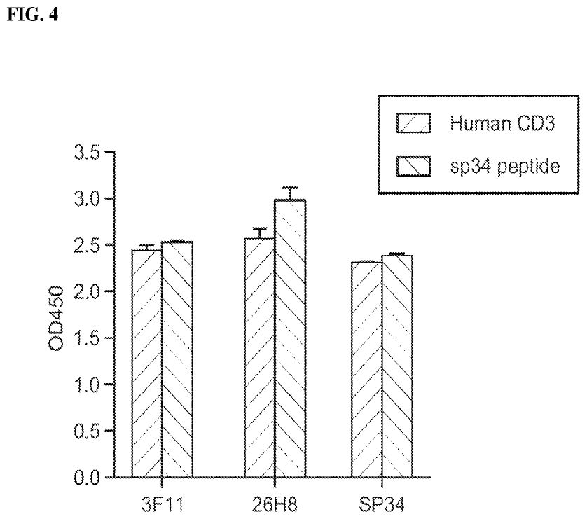

After fusion, cells were selected in complete DMEM medium containing hypoxanthine/aminopterin/thymidine (Thermo Fisher Scientific) and 10% UltraCruz® Hybridoma Cloning Supplement (Santa Cruz) for 10-12 days. Hybridoma culture supernatants were screened for reacting to human CD3$/CD3γ proteins. For screening with ELISAs 50 μL of human CD3 proteins diluted in carbonate/bicarbonate buffer, pH 9.6 at the concentration of 1 μg/mL were added into micro-wells and incubated at 4° C. overnight. Wells were washed with PBS containing 0.1% Tween 20 (PBST) three times and blocked with 200 μL of PBS containing 1% bovine serum albumin. After incubation at room temperature for 1 h and wash with PBST three times, 50 μL of culture supernatants were added into wells and incubated at room temperature for 1 hr. After wash, 50 μL of 1:10,000 diluted goat anti-mouse IgG.Fc-HRP (Jackson ImmunoResearch) were added into wells and incubated at room temperature for 1 hr. After wash six times, 50 μL of TMB substrates (Thermo Fisher Scientific) were added into wells for color development. The reactions were stopped by adding 50 μL of 1N HCl. Wells were read at optical density 450 nm with a Synergy 4 microplate reader (Biotek). After two individual screenings, hybridoma clones with different binding signals were identified (FIG. 3). The reactivity towards the synthetic sp34 peptide (seq. 03, CSBio) which is the epitope of MAb SP34, a monoclonal antibody reacting to both human and cynomolgus CD3$ chains, was further tested. The pyroGlu indicates the pyroglutamate (seq. 03). In ELISA analyses Clone 3F11 and 26H8 were shown to react to sp34 peptides (FIG. 4).

### T Cell Binding and Activation Analyses

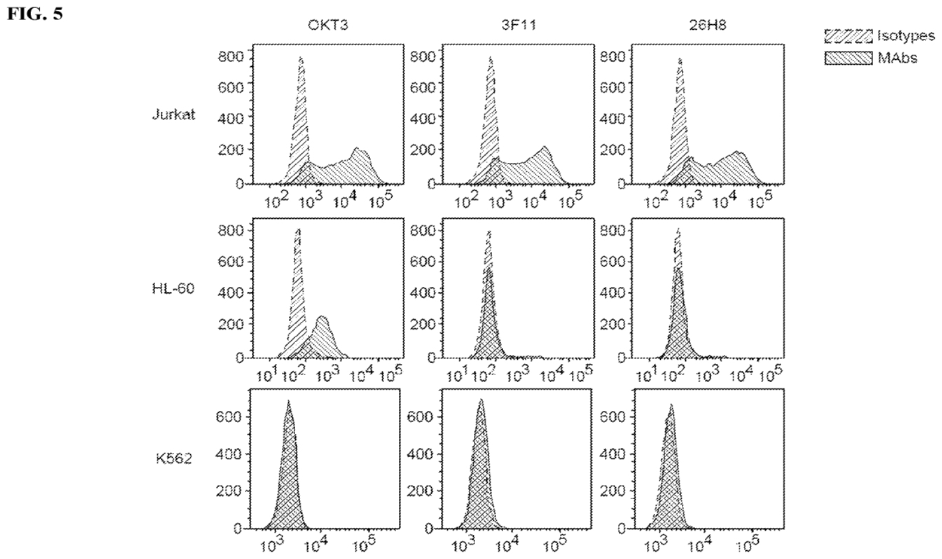

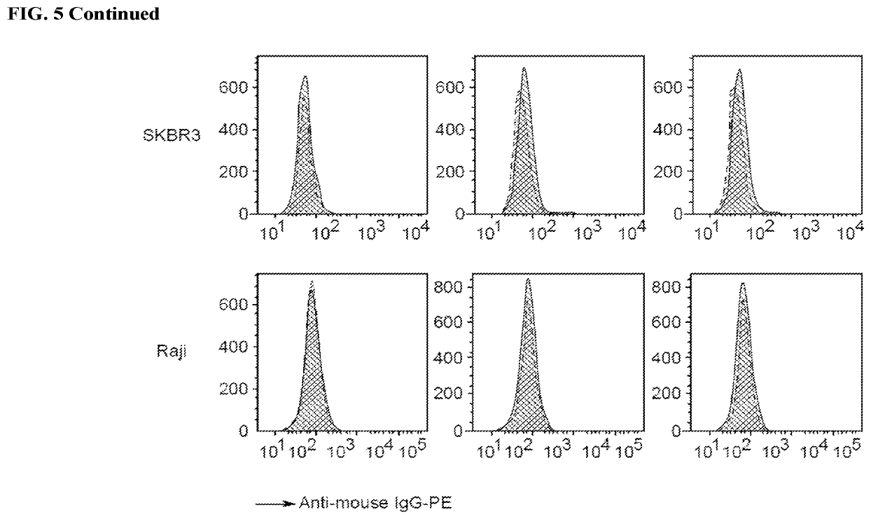

Hybridoma culture supernatants were tested for surface binding for various human cell lines, Jurkat, HL-60, K562, SKBR3, and Raji (ATCC) using flow cytometry analyses. One million cells were incubated with 100 μL of culture supernatants on ice for 30 min and washed with cold PBS containing 1% FBS and 0.1% sodium azide. Cells were then incubated with 100 μL of 1:400 diluted AffiniPure goat anti-mouse IgG.Fc-AF488 (Jackson ImmunoResearch) on ice for 30 min. Cells were washed with cold PBS containing 1% FBS and 0.1% sodium azide and then analyzed by using an Accuri C6 flow cytometer (BD). The three identified anti-CD3 clones were able to bind to Jurkat cells without reacting to other CD3 negative cell lines (FIG. 5). Mouse OKT3 (BioLegend) was used as a positive control.

To test T cell receptor activation by these three antibodies, 5×105 Jurkat cells were resuspended in 500 μL of complete RPMI medium mixed with 500 μL of each hybridoma culture supernatant. After incubating at 37° C. for one hour, the secondary antibodies using AffiniPure goat F(ab′)₂ anti-mouse IgG.Fc (Jackson ImmunoResearch) at the final concentration of 10 μg/mL were added into cells to cross-link anti-CD3 antibodies. After culturing for one day, cells were centrifuged and stained with mouse anti-human CD69-PE (Biolegend). After wash, cells were analyzed by a flow cytometer. Clone 3F11 and 26H8 slightly stimulated Jurkat cell activation with antibodies alone and significantly activated CD69 expression in the presence of cross-linking antibodies (FIG. 6)

### Isotyping and Cloning of VH/VL of Anti-CD3 Antibodies

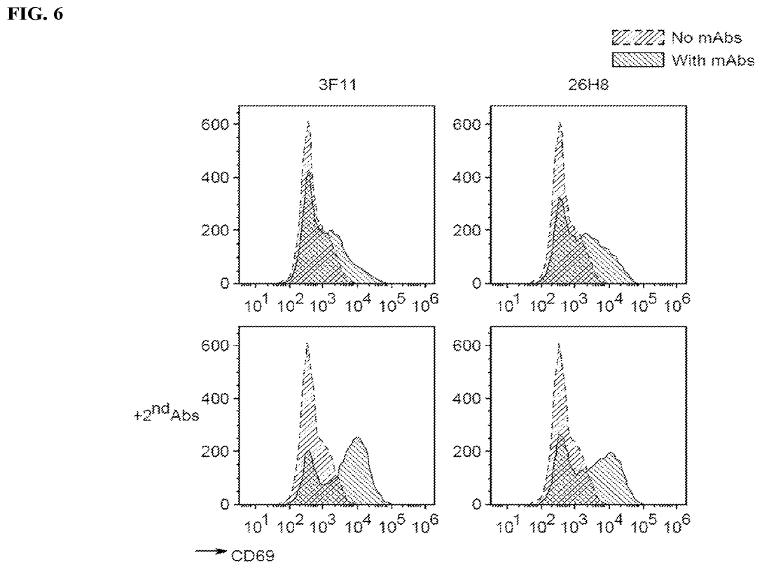

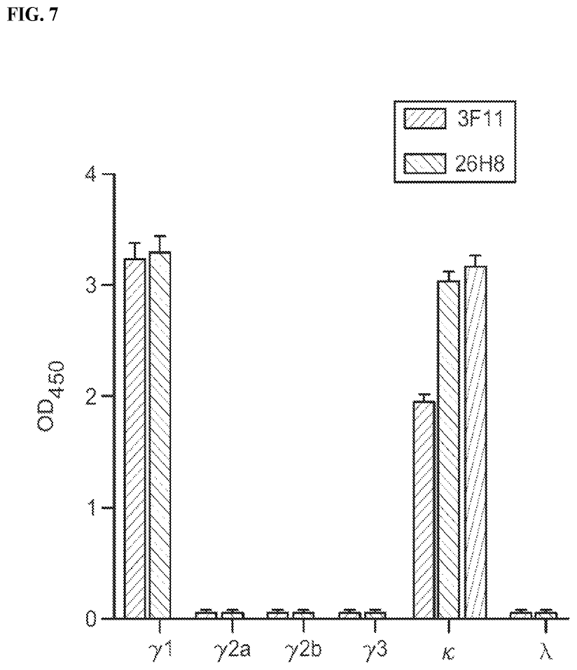

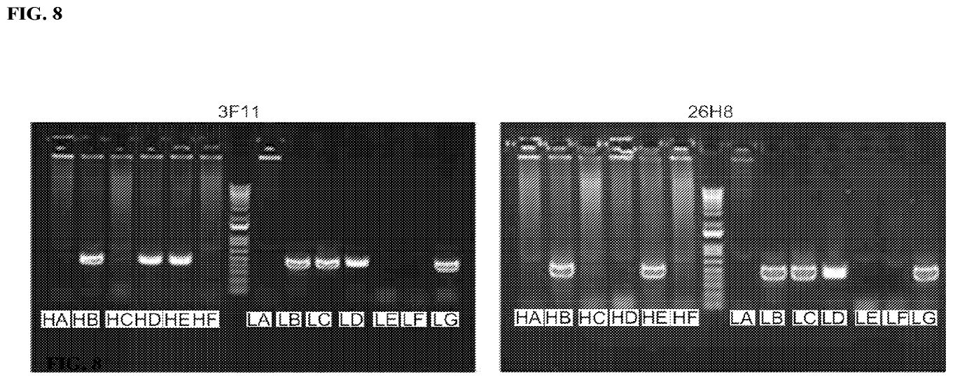

To determine the Ig isotypes of mouse antibodies, sp34 peptides were used as antigens coated onto micro-wells for incubation with hybridoma culture supernatants in ELISAs. HRP-conjugated goat anti-mouse Igγ1, Igγ2a, Igγ2b, Igγ3, Igκ and Igλ (SouthemBiotech) were used to detect antibody isotypes and to develop colors with TMB substrates. All of these three antibodies were of yl and kappa isotypes (FIG. 7). To clone VH/VL sequences from hybridomas, mRNAs were extracted using a Quick-RNA Microprep kit (Zymo Research). First-strand cDNAs were synthesized using a SuperScript III First-Strand Synthesis System (Thermo Fisher Scientific). The VH and VL fragments were amplified by PCRs using a Mouse Ig-Primer Set (Millipore Sigma) and OneTaq 2× Master Mix (NEB). Amplified DNA fragments were purified using a DNA Clean-up kit (Zymo Research) and ligated into pGEM-T vectors (Promega) for sequencing (FIG. 8). Encoded amino acid sequences of VH and VL for 3F11 and 26H8 were listed, respectively (Seq. 04-07).

### Human T Cell Activation and Proliferation Assays

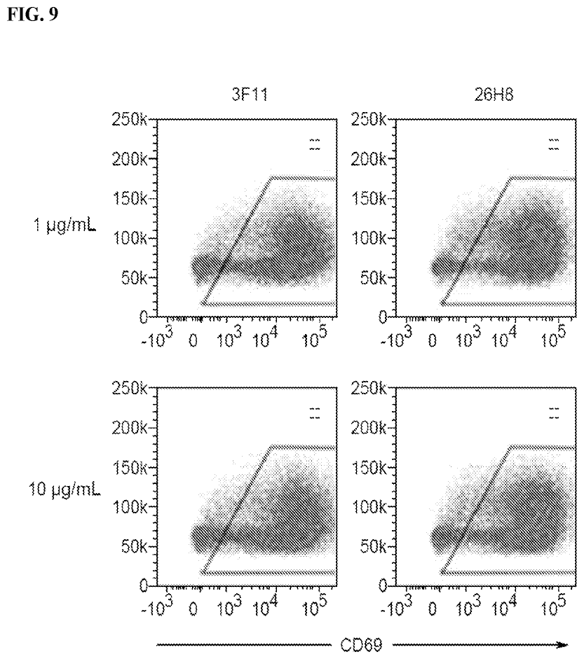

Human peripheral blood mononuclear cells (PBMCs) were isolated from leukocyte-rich fractions acquired from the blood and platelet center of City of Hope. Leukocytes were fractionated using Ficoll-paque plus (GE healthcare) and SepMate 50 tubes (STEMCELL technology). Cells were spun with 1,200×g for 10 min. PBMC fractions were transferred and washed with PBS two times by centrifugation. To test human T cell activation, two million PBMCs were cultured in fresh medium containing 1 or 10 μg/mL of anti-CD3 antibodies and 5 μg/mL of anti-CD28 antibodies (BioLegend) for 2 days. Cells were then centrifuged and resuspended in FACS buffers. PE-conjugated anti-CD69 antibodies (BioLegend) were used for staining. After washing, cells were analyzed by FACS. Results showed that 26H8 induce extensive CD69 expression (FIG. 9).

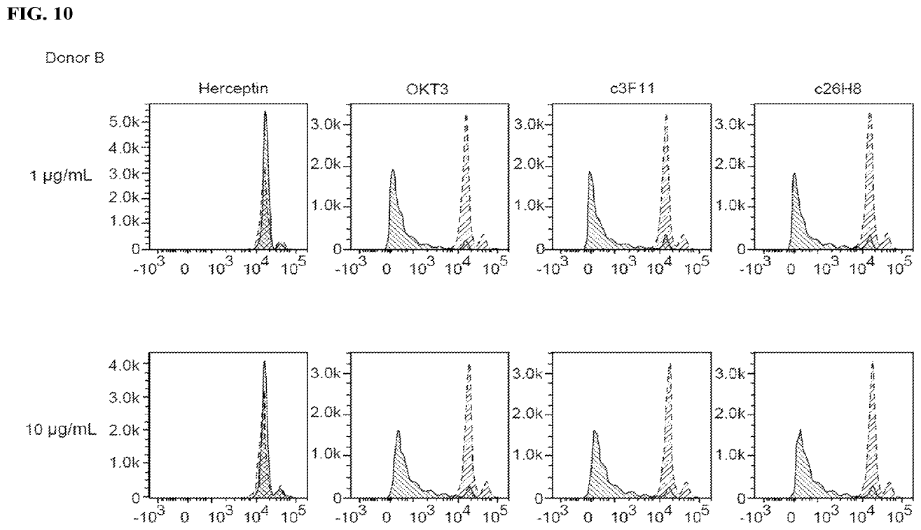

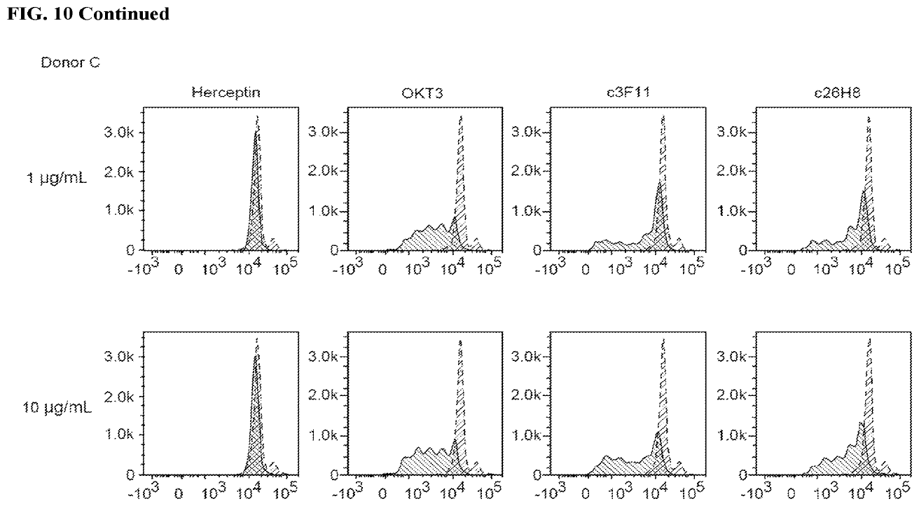

To measure cell proliferation, cells were first labelled using a CellTrace Far Red kit (ThermoFisher). Ten million cells were resuspeded in 1 mL of 1 μM CellTrace Far Red dye at 37° C. for 20 minutes and then inactivated by adding 5 mL of culture medium and incubating for 5 minutes. Cells were then centrifuged to remove the medium and resuspended with fresh medium containing 1 or 10 μg/mL of anti-CD3 antibodies and 5 μg/mL of anti-CD28 antibodies at the concentration of 2×106 cells/mL. Cells were cultured for three days and replenished with fresh medium for culturing two more days. Cells were spun to remove the medium and resuspended in FACS buffers for flow cytometric analysis. Results showed that Clone 3F11 and 26H8 induced similar cell proliferation profiles with OKT3 (FIG. 10).

### Cytokine ELISA

IL-2 and IFN-γ were measured by using LEGEND MAX Human IL-2 and IFNγ ELISA kits (BioLegend), respectively. Two million PBMCs were resuspended in 1 mL fresh medium containing 1 μg/mL of anti-CD3 antibodies with or without 5 μg/mL of anti-CD28 antibodies and cultured for 3 days. Cell culture supernatants were spun at 800×g for 5 min and transferred for ELISA analysis. The ELISA procedures were followed according to the manufacturer's manuals. In brief, supernatants were added into capture antibody-coated wells and incubated at room temperature for 2 hr. After washing with PBST three times, detection antibodies were added into wells and incubated at room temperature for 1 hr. Wells were washed three times and loaded with diluted Streptavidin-HRP for incubating 30 min. After washing six times, the substrate solution was added for enzymatic reaction and color development followed by adding 1N HCl for reaction termination. Wells were read at optical density 450 nm. The results showed that 3F11 and 26H8 stimulated human T cells producing IL-2 and IFN-γ (FIG. 11).

Affinity Measurement with Biacore

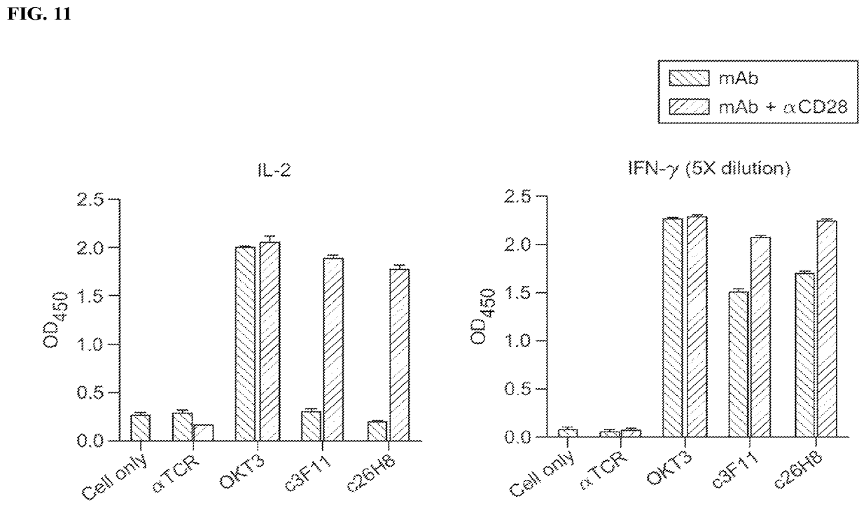

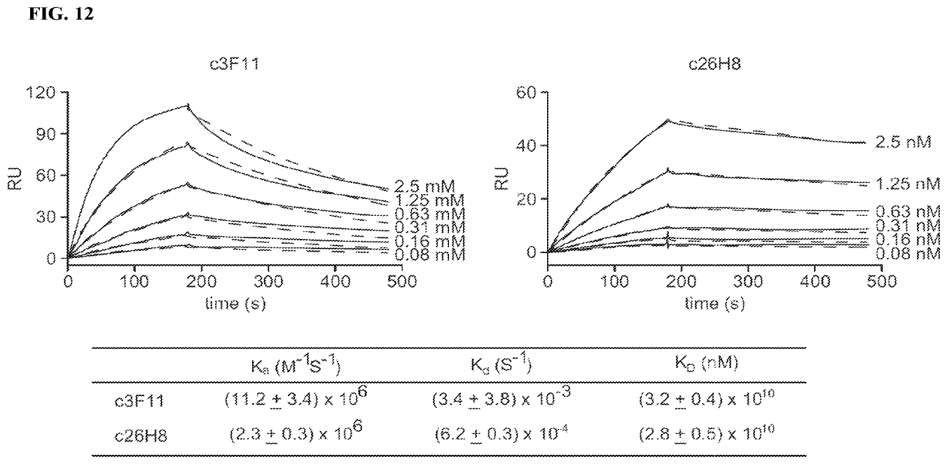

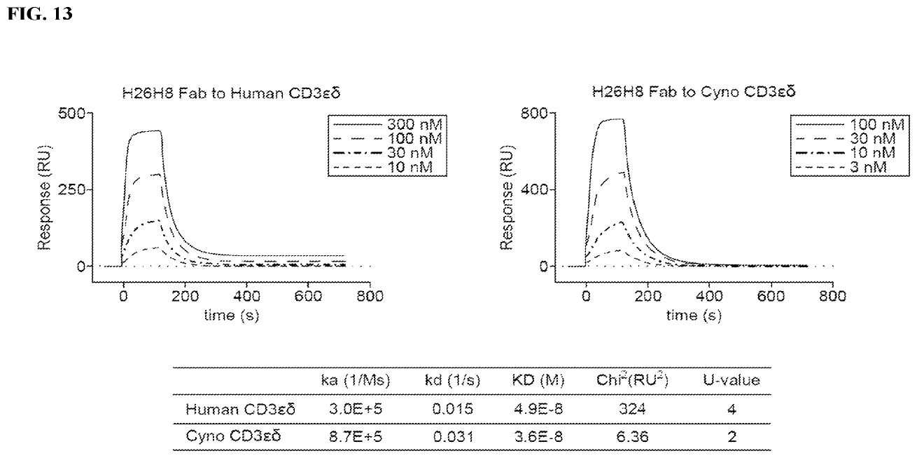

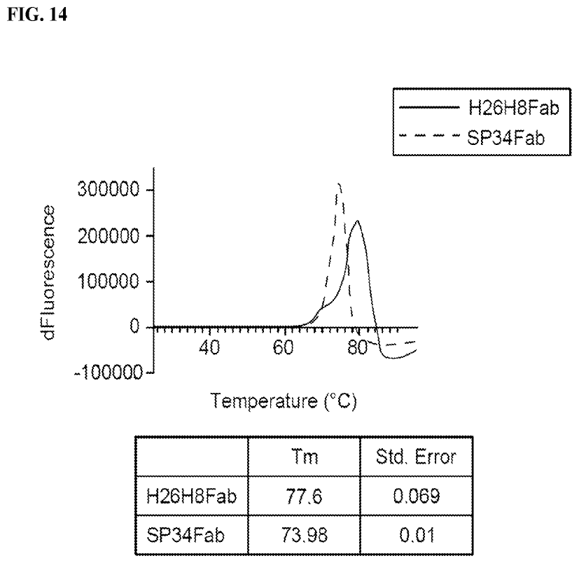

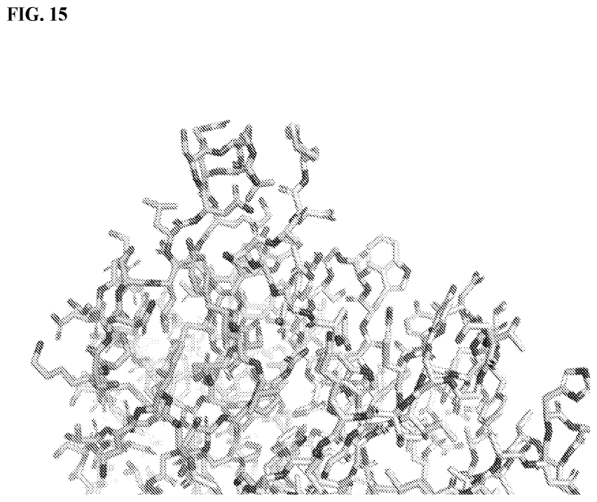

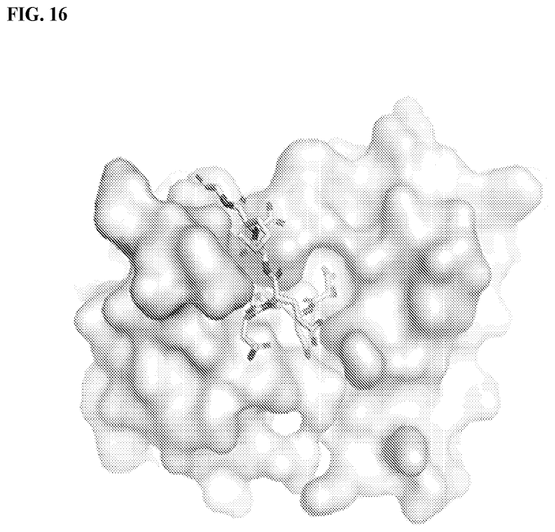

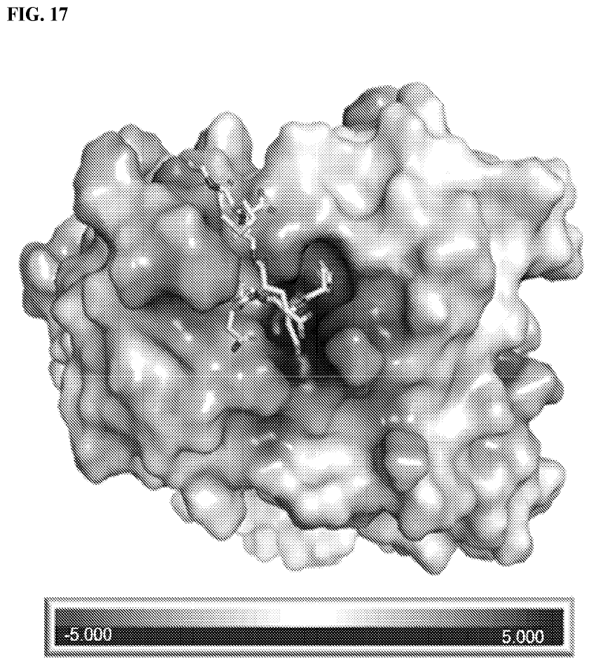

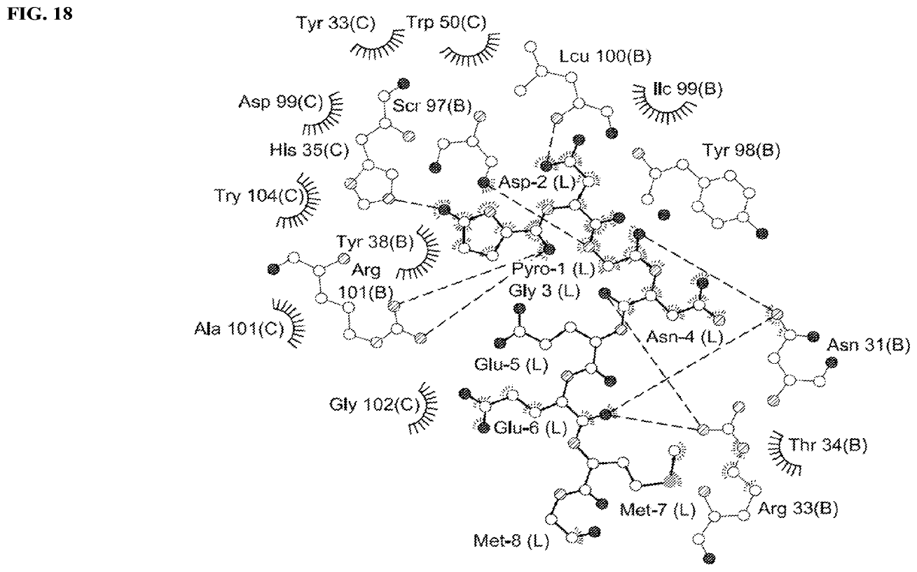

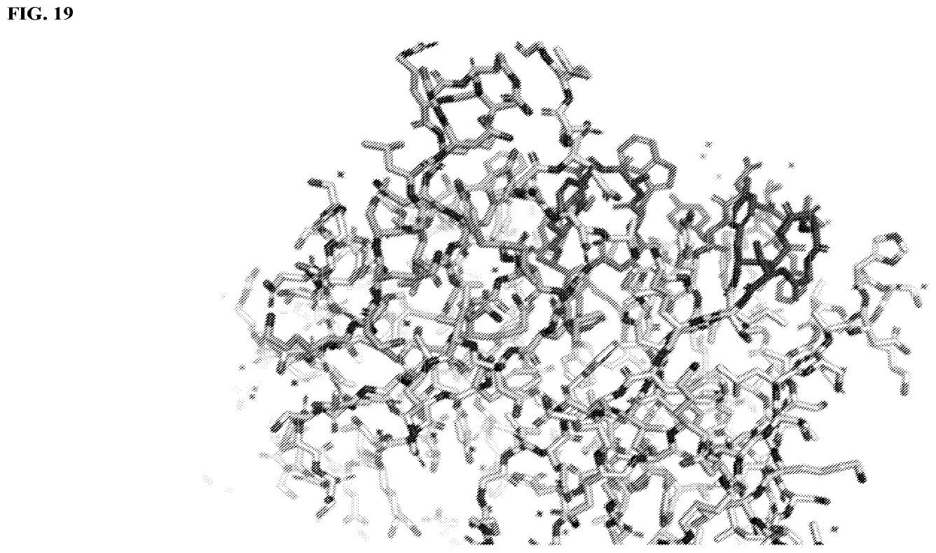

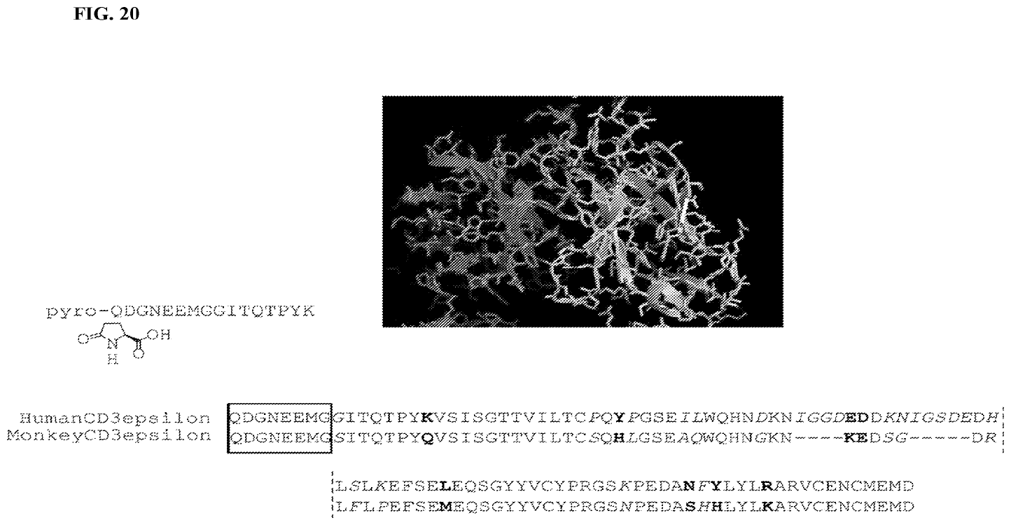

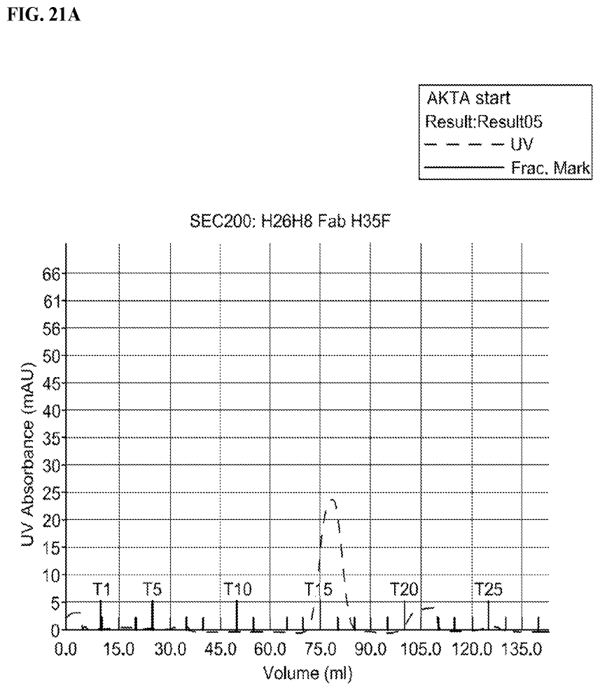

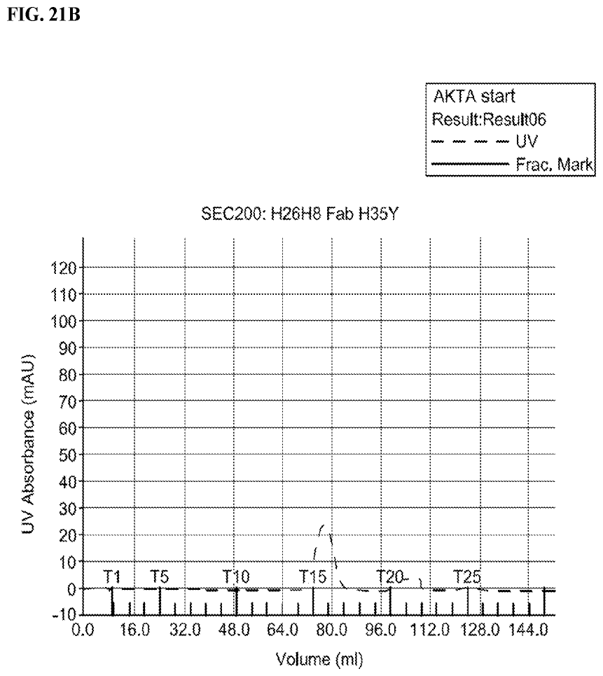

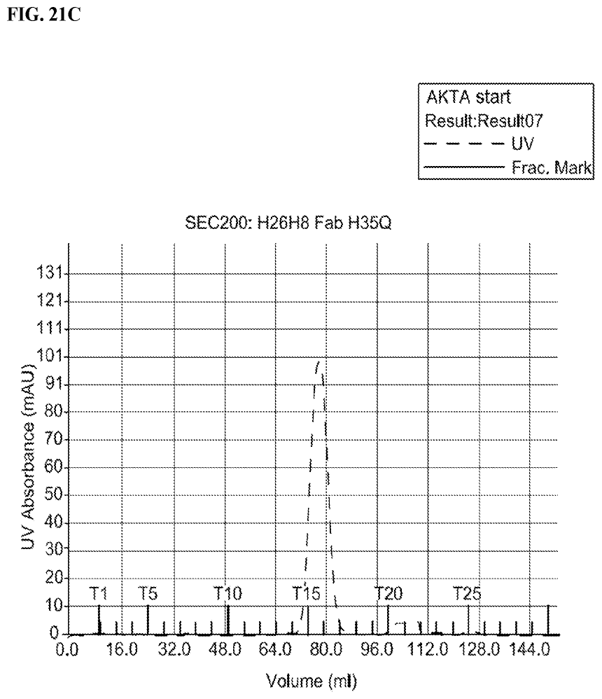

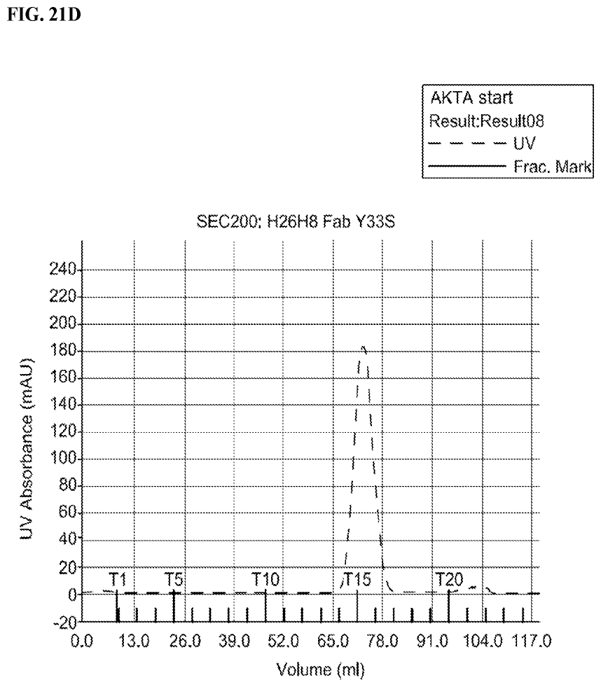

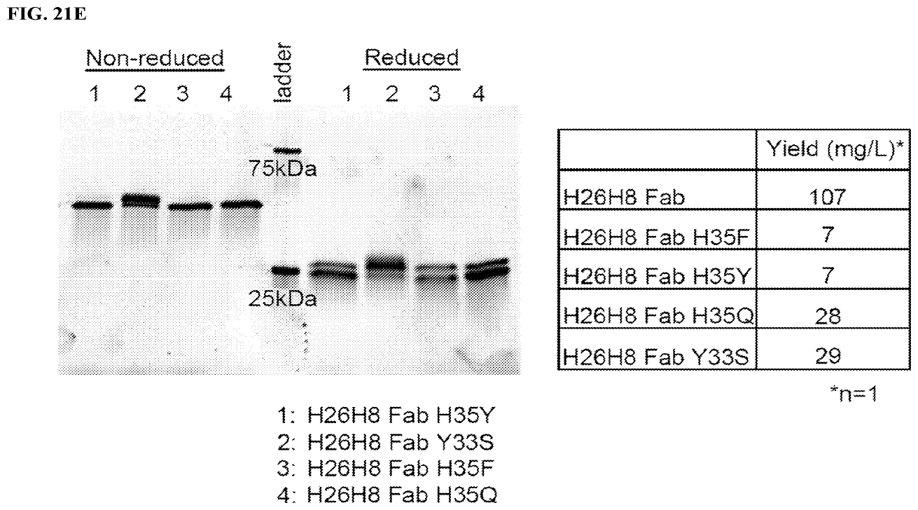

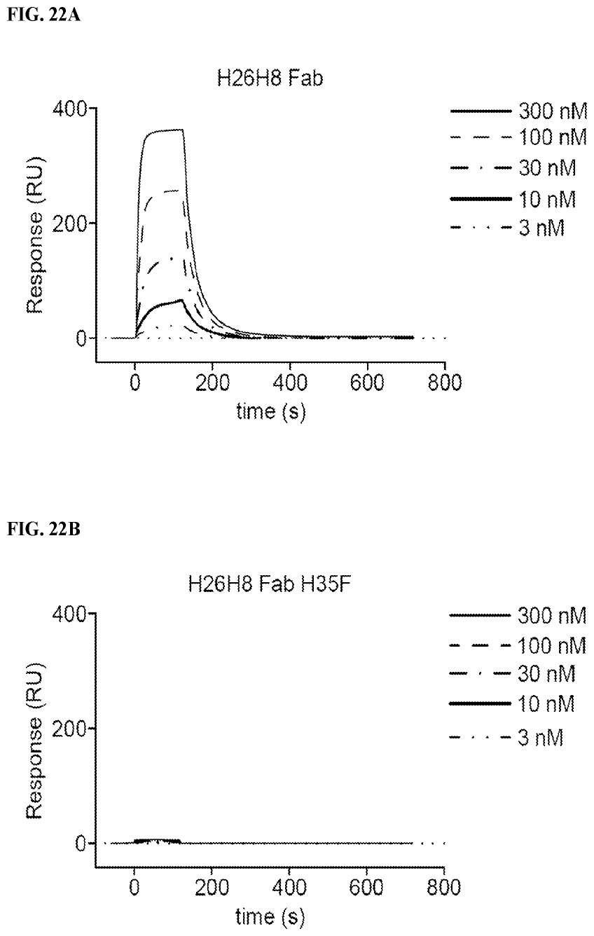

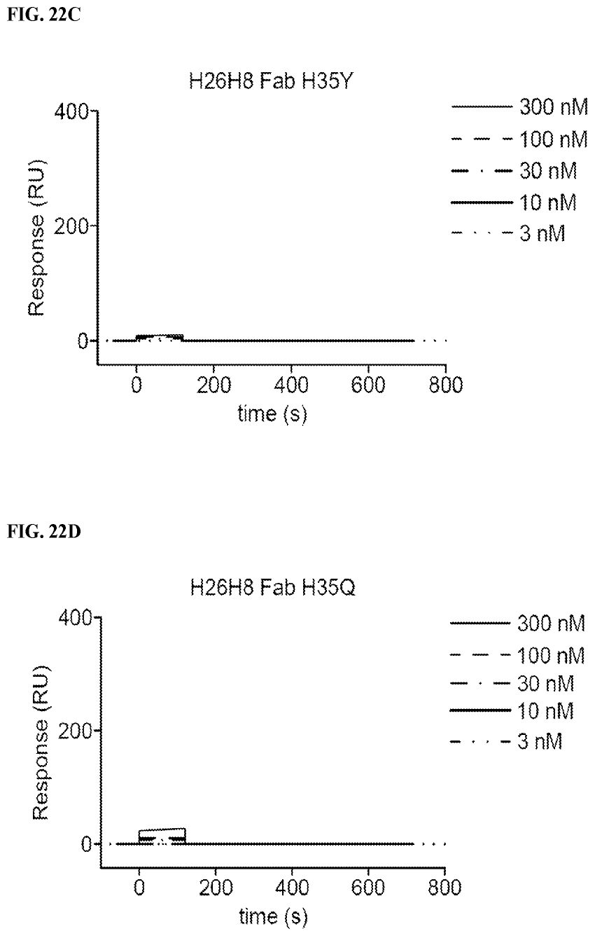

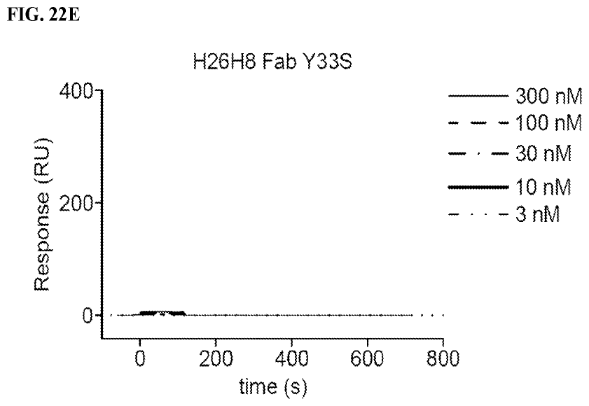

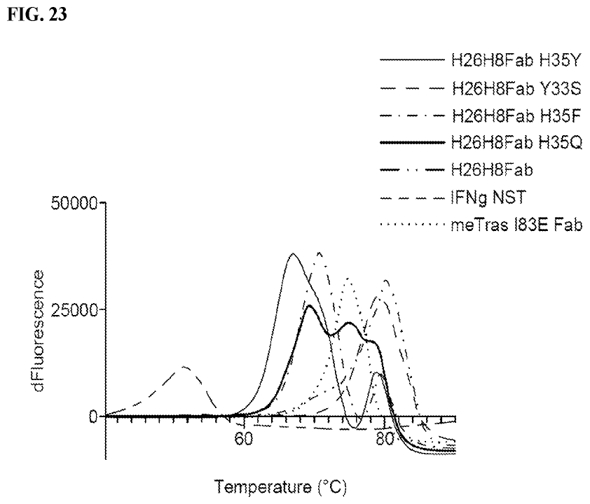

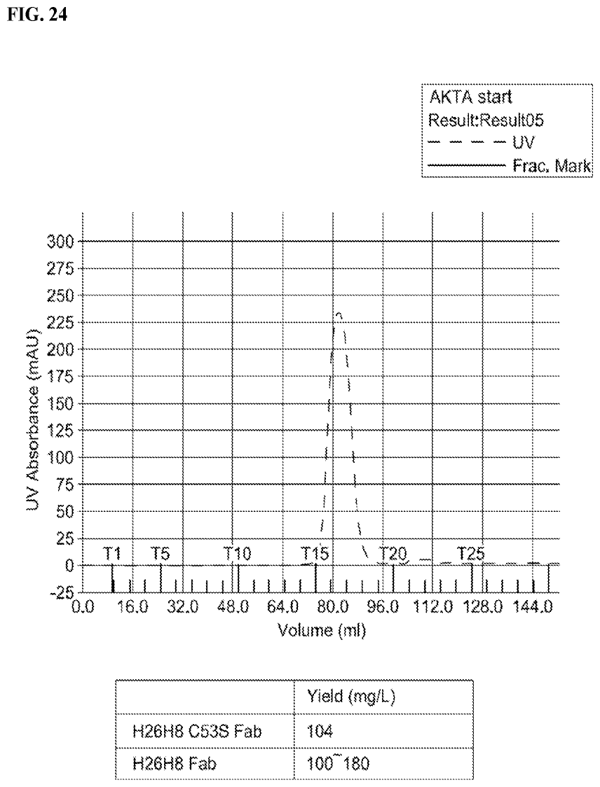

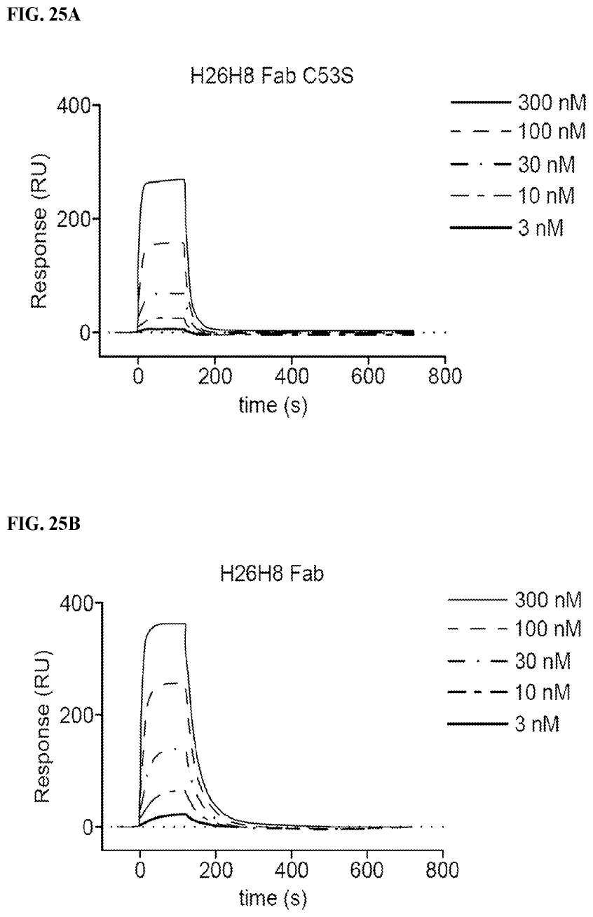

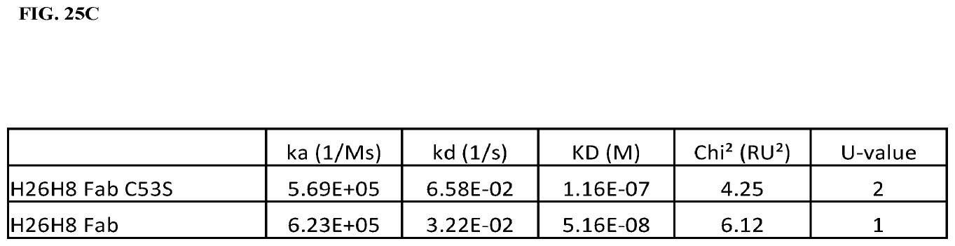

Binding affinities for the different antibodies of interest were measured by surface plasmon resonance (SPR), using a Biacore T200 (GE Healthcare). Human CD3-IgA was immobilized to 200 RU on a CM5 (GE Life Sciences) chip using NHS chemistry. Analytes (CD3 antibodies) were diluted 2× in concentrations (from 12.5 nM to 0.125 nM) and flown over chip at a flow rate of 30 □l/min with HBS-EP+ buffer. Data were analyzed using Biacore T200 Evaluation software v3.0. The results were shown in FIG. 12.

Alignment of SP34 VL (SEQ ID NO:37) md VH (SEQ ID NO:38) with anti-CD3 antibody clone 3F11 VL (SEQ ID NO:11) and VH (SEQ ID NO: 12) and clone 261H8 VL (SEQ ID NO:9) and VH (SEQ ID NO: 10).

| Variable Light (VL) chains: | |
|---|---|
| 34 | QAVV-TQEPSLTVSPGGTVTLTCGSSTGA---VITSNYANWVQQKPGKSPRGLIGGTNKR |
| 3F11 | DIVMSQSPSSLAVSAGEKVTISCKSSQSLLNNRTRKNYLAWYQQKPGOSPKLLIYWASTR |
| 26H8 | DIVMSQSPSSLAVSAGEKVTMSCKSSQSLLNSRTRKNYLAWYQQKPGQSPKLLIYWASTR |
| | : *: **:** * .**::* ** . * .** * *****:**: ** :..* |
| 34 | APGVPARFSGSLLGGKAALTISGAQPEDEADYYCALWYSNHWVFGGGTKLTVL |
| 3F11 | ESGVPDRFTGSGSGTDFTLTISSVQAEDLAVYYCKQSYIL-RTFGGGTKLEIK |
| 26H8 | ESGVPDRFTGSGSGTDFTLTISSVOAEDLAVYYCKQSYIL-RTFGGGTKLEIK |
| | *** **:** * . :***...* ** * *** * .******* : |
| Variable Heavy (VI) Chains: | |
| 34 | EVQLVESGGGLVQPGGSLRLSCAASGFTESTYAMNWVRQAPGKGLEWVGRIRSKYNNYAT |
| 3F11 | QVQLQQSGPDLVRPGASVKMSCKASGYTFTNYYVHWLNQRPGQGLEWIGWIYPG--DGST |
| 26H8 | QVQLQQSGPELVKPGASVPMSCKASGYTFTNYYIHWVRQRPGQGLEWIGWIYCG--DGST |
| | :*** :** **:**.*:::** :**:**:.* ::*:.* **:****:* * : * |
| 34 | YYADSVKGRETISRDDSKNTLYLQMNSLRAEDTAVYYCVRHGNFGDSYVSWFAYWGQGTL |
| 3F11 | KFNEKFKGKTTLTADKSSSTAYILLSSLTSADSAIYFCTRD--A----GYYFDVWGAGTT |
| 26H8 | KFNEQFKDRTTLTADRSSSTAYMELSSLTSEDSAIYFCARDWAG----SYFFDEWGQGTT |
| | : :..*.: *:: *.*..* *: :.** : *:*:*:*.*. :* ** ** |
| 34 | VTVSS |
| 3F11 | VTVSS |
| 26H8 | LTVSS |
| | :**** |

TABLE 1
Data collection and refinement statistics -
humanized 26H8 and CD3 peptide

| Resolution range | 34.8-2.7 | (2.796-2.7) |
|---|---|---|
| Space group | P 21 2 21 | |
| Unit cell (x, y, z, a, b, c) | 62.17 Å, 91.22 Å, 107.64 Å, | |
| | 90°, 90°, 90° | |
| Total reflections | 113501 | (10874) |
| Unique reflections | 17260 | (1664) |
| Multiplicity | 6.6 | (6.5) |
| Completeness (%) | 99.12 | (99.16) |
| Mean I/sigma(I) | 12.91 | (2.52) |
| Wilson B-factor | 41.86 | |
| R-merge | 0.131 | (0.7473) |
| R-meas | 0.1423 | (0.8126) |
| R-pim | 0.05477 | (0.3151) |
| CC1/2 | 0.997 | (0.841) |
| CC* | 0.999 | (0.956) |
| Reflections used in refinement | 17251 | (1662) |
| Reflections used for R-free | 842 | (69) |
| R-work | 0.2043 | (0.2679) |
| R-free | 0.2537 | (0.3107) |
| CC(work) | 0.948 | (0.882) |
| CC(free) | 0.887 | (0.821) |

| | |
|---|---|
| Number of non-hydrogen atoms | 3483 |
| macromolecules | 3438 |
| ligands | 0 |
| solvent | 45 |
| Protein residues | 444 |
| RMS(bonds) | 0.008 |
| RMS(angles) | 1.09 |
| Ramachandran favored (%) | 91.26 |
| Ramachandran allowed (%) | 7.59 |
| Ramachandran outliers (%) | 1.15 |
| Rotamer outliers (%) | 10.74 |
| Clashscore | 10.42 |
| Average B-factor | 62.82 |
| macromolecules | 63.08 |
| solvent | 43.13 |
| Number of TLS groups | 5 |

### P Embodiments

P Embodiment 1. An anti-CD3 antibody comprising a light chain variable domain and a heavy chain variable domain, wherein said light chain variable domain comprises: a CDR L1 as set forth in SEQ ID NO: 1, a CDR L2 as set forth in SEQ ID NO:2 and a CDR L3 as set forth in SEQ ID NO:3; and wherein said heavy chain variable domain comprises: a CDR H1 as set forth in SEQ ID NO:4, a CDR H2 as set forth in SEQ ID NO:5, and a CDR H3 as set forth in SEQ ID NO: 6.

P Embodiment 2. The anti-CD3 antibody of P embodiment 1, wherein said antibody is a humanized antibody.

P Embodiment 3. The anti-CD3 antibody of P embodiment 1, wherein said antibody is a chimeric antibody.

P Embodiment 4. The anti-CD3 antibody of P embodiment 1, wherein said antibody is a Fab′ fragment.

P Embodiment 5. The anti-CD3 antibody of P embodiment 1, wherein said antibody is a single chain antibody (scFv).

P Embodiment 6. The anti-CD3 antibody of P embodiment 1, wherein said light chain variable domain comprises the sequence of SEQ ID NO:7, SEQ ID NO:9, or SEQ ID NO:11.

P Embodiment 7. The anti-CD3 antibody of P embodiment 1, wherein said heavy chain variable domain comprises the sequence of SEQ ID NO:8, SEQ ID NO:10, or SEQ ID NO:12.

P Embodiment 8. The anti-CD3 antibody of P embodiment 1, wherein said light chain variable domain comprises the sequence of SEQ ID NO:7 and said heavy chain variable domain comprises the sequence of SEQ ID NO:8.

P Embodiment 9. The anti-CD3 antibody of P embodiment 1, wherein said light chain variable domain comprises the sequence of SEQ ID NO:9 and said heavy chain variable domain comprises the sequence of SEQ ID NO:10.

P Embodiment 10. The anti-CD3 antibody of P embodiment 1, wherein said light chain variable domain comprises the sequence of SEQ ID NO:11 and said heavy chain variable domain comprises the sequence of SEQ ID NO:12.

P Embodiment 11. The anti-CD3 antibody of any one of P embodiments 1-10, wherein said antibody is bound to a CD3 protein.

P Embodiment 12. The anti-CD3 antibody of P embodiment 11, wherein said CD3 protein is a human CD3 protein.

P Embodiment 13. The anti-CD3 antibody of P embodiment 11, wherein said CD3 protein is a non-human primate CD3 protein.

P Embodiment 14. The anti-CD3 antibody of any one of P embodiments 11-14, wherein said CD3 protein forms part of a cell.

P Embodiment 15. The anti-CD3 antibody of P embodiment 14, wherein said CD3 protein is expressed on the surface of the cell.

P Embodiment 16. The anti-CD3 antibody of P embodiment 14, wherein said cell is a T cell.

P Embodiment 17. The anti-CD3 antibody of any one of P embodiments 1-16, wherein said antibody is bound to a therapeutic moiety or a diagnostic moiety.

P Embodiment 18. A recombinant protein comprising: (i) a first antibody region capable of binding a target cell ligand; and (ii) a second antibody region, comprising: (a) a second light chain variable domain comprising a CDR L1 as set forth in SEQ ID NO:1, a CDR L2 as set forth in SEQ ID NO:2 and a CDR L3 as set forth in SEQ ID NO:3; and (b) a second heavy chain variable domain comprising a CDR H1 as set forth in SEQ ID NO:4, a CDR H2 as set forth in SEQ ID NO:5, and a CDR H3 as set forth in SEQ ID NO: 6.

P Embodiment 19. The recombinant protein of P embodiment 18, wherein said second light chain variable domain comprises the sequence of SEQ ID NO:7, SEQ ID NO:9, or SEQ ID NO:11; and wherein said second heavy chain variable domain comprises the sequence of SEQ ID NO: 8, SEQ ID NO:10, or SEQ ID NO:12.

P Embodiment 20. The recombinant protein of P embodiment 18, wherein said second light chain variable domain comprises the sequence of SEQ ID NO:7 and said second heavy chain variable domain comprises the sequence of SEQ ID NO: 8.

P Embodiment 21. The recombinant protein of P embodiment 18, wherein said second light chain variable domain comprises the sequence of SEQ ID NO:9 and said second heavy chain variable domain comprises the sequence of SEQ ID NO: 10.

P Embodiment 22. The recombinant protein of P embodiment 18, wherein said second light chain variable domain comprises the sequence of SEQ ID NO:11 and said second heavy chain variable domain comprises the sequence of SEQ ID NO:12.

P Embodiment 23. The recombinant protein of any one of P embodiments 18-22, wherein said first antibody region is bound to the target cell ligand.

P Embodiment 24. The recombinant protein of any one of P embodiments 18-23, wherein said target cell ligand is a cancer protein or fragment thereof.

P Embodiment 25. The recombinant protein of any one of P embodiments 18-24, wherein said target cell ligand forms part of a cell.

P Embodiment 26. The recombinant protein of P embodiment 25, wherein said cell is a cancer cell.

P Embodiment 27. The recombinant protein of any one of P embodiments 18-26, wherein said second antibody region is bound to a CD3 protein.

P Embodiment 28. The recombinant protein of P embodiment 27, wherein said CD3 protein is a human CD3 protein.

P Embodiment 29. The recombinant protein of P embodiment 27, wherein said CD3 protein is a non-human primate CD3 protein.

P Embodiment 30. The recombinant protein of any one of P embodiments 27-29, wherein said CD3 protein forms part of a cell.

P Embodiment 31. The recombinant protein of any one of P embodiments 27-30, wherein said CD3 protein is expressed on the surface of a cell.

P Embodiment 32. The recombinant protein of P embodiment 31, wherein said cell is a T cell.

P Embodiment 33. The recombinant protein of any one of P embodiments 18-32, wherein said recombinant protein is bound to a therapeutic moiety or a diagnostic moiety.

P Embodiment 34. The recombinant protein of any one of P embodiments 18-33, wherein said first antibody region is a first Fab′ fragment or said second antibody region is a second Fab′ fragment.

P Embodiment 35. The recombinant protein of any one of P embodiments 18-33, wherein said first antibody region is a single chain variable fragment (scFv) or said second antibody region is a second single chain variable fragment (scFv).

P Embodiment 36. A pharmaceutical composition comprising (i) a therapeutically effective amount of an anti-CD3 antibody of any one of P embodiments 1-17, or a therapeutically effective amount of a recombinant protein of any one of P embodiments 18-35 and (ii) a pharmaceutically acceptable excipient.

P Embodiment 37. A method of treating cancer in a subject in need thereof, said method comprising administering to a subject a therapeutically effective amount of an anti-CD3 antibody of any one of P embodiments 1-17 or a therapeutically effective amount of a recombinant protein of any one of P embodiments 18-35, thereby treating cancer in said subject.

P Embodiment 38. An isolated nucleic acid encoding an anti-CD3 antibody f any one of P embodiments 1-17.

P Embodiment 39. A cell including an anti-CD3 antibody of any one of P embodiments 1-17.

P Embodiment 40. A method of forming an anti-CD3 antibody, said method comprising administering a polypeptide comprising the sequence of SEQ ID NO:14 or SEQ ID NO:15 to a mammal, thereby forming an anti-CD3 antibody.

P Embodiment 41. The method of P embodiment 40, further comprising administering an adjuvant to said mammal.

### Embodiments

Embodiment 1. An anti-CD3 antibody comprising a light chain variable domain and a heavy chain variable domain, wherein said light chain variable domain comprises: a CDR L1 as set forth in SEQ ID NO:1, a CDR L2 as set forth in SEQ ID NO:2 and a CDR L3 as set forth in SEQ ID NO:3; and wherein said heavy chain variable domain comprises: a CDR H1 as set forth in SEQ ID NO:4, a CDR H2 as set forth in SEQ ID NO:5, and a CDR H3 as set forth in SEQ ID NO:6.

Embodiment 2. An anti-CD3 antibody comprising a light chain variable domain and a heavy chain variable domain, wherein said light chain variable domain comprises: a CDR L1 as set forth in SEQ ID NO:1, a CDR L2 as set forth in SEQ ID NO:2 and a CDR L3 as set forth in SEQ ID NO:3; and wherein said heavy chain variable domain comprises: a CDR H1 as set forth in SEQ ID NO:4, a CDR H2 as set forth in SEQ ID NO: 16, and a CDR H3 as set forth in SEQ ID NO: 6.

Embodiment 3. An anti-CD3 antibody comprising a light chain variable domain and a heavy chain variable domain, wherein said light chain variable domain comprises: a CDR L1 as set forth in SEQ ID NO:1, a CDR L2 as set forth in SEQ ID NO:2 and a CDR L3 as set forth in SEQ ID NO:3; and wherein said heavy chain variable domain comprises: a CDR H1 as set forth in SEQ ID NO:4, a CDR H2 as set forth in SEQ ID NO: 19, and a CDR H3 as set forth in SEQ ID NO:20.

Embodiment 4. The anti-CD3 antibody of any one of embodiments 1-3, wherein said antibody is a humanized antibody.

Embodiment 5. The anti-CD3 antibody of any one of embodiments 1-3, wherein said antibody is a chimeric antibody.

Embodiment 6. The anti-CD3 antibody of any one of embodiments 1-5, wherein said antibody is a Fab′ fragment.

Embodiment 7. The anti-CD3 antibody of any one of embodiments 1-5, wherein said antibody is a single chain antibody (scFv).

Embodiment 8. The anti-CD3 antibody of any one of embodiments 1-7, wherein said light chain variable domain comprises the sequence of SEQ ID NO:7, SEQ ID NO:9, or SEQ ID NO:11.

Embodiment 9. The anti-CD3 antibody of any one of embodiments 1-8, wherein said heavy chain variable domain comprises the sequence of SEQ ID NO:8, SEQ ID NO:10, SEQ ID NO:12, or SEQ ID NO:17.

Embodiment 10. The anti-CD3 antibody of embodiment 1, wherein said heavy chain variable domain comprises a serine at a position corresponding to position 53 of SEQ ID NO:8.

Embodiment 11. The anti-CD3 antibody of any one of embodiments 1, 4, or 6-9, wherein said light chain variable domain comprises the sequence of SEQ ID NO:7 and said heavy chain variable domain comprises the sequence of SEQ ID NO:8.

Embodiment 12. The anti-CD3 antibody of any one of embodiments 1 or 5-9, wherein said light chain variable domain comprises the sequence of SEQ ID NO:9 and said heavy chain variable domain comprises the sequence of SEQ ID NO:10.

Embodiment 13. The anti-CD3 antibody of any one of embodiments 2, 4, or 6-9, wherein said light chain variable domain comprises the sequence of SEQ ID NO:7 and said heavy chain variable domain comprises the sequence of SEQ ID NO:17.

Embodiment 14. The anti-CD3 antibody of any one of embodiments 3 or 5-9, wherein said light chain variable domain comprises the sequence of SEQ ID NO:11 and said heavy chain variable domain comprises the sequence of SEQ ID NO:12.

Embodiment 15. The anti-CD3 antibody of any one of embodiments 1-14, wherein said antibody is bound to a CD3 protein.

Embodiment 16. The anti-CD3 antibody of embodiment 15, wherein said CD3 protein is a human CD3 protein.

Embodiment 17. The anti-CD3 antibody of embodiment 15, wherein said CD3 protein is a non-human primate CD3 protein.

Embodiment 18. The anti-CD3 antibody of any one of embodiments 15-18, wherein said CD3 protein forms part of a cell.

Embodiment 19. The anti-CD3 antibody of embodiment 18, wherein said CD3 protein is expressed on the surface of said cell.

Embodiment 20. The anti-CD3 antibody of embodiment 18, wherein said cell is a T cell.

Embodiment 21. The anti-CD3 antibody of any one of embodiments 1-20, wherein said antibody is bound to a therapeutic moiety or a diagnostic moiety.

Embodiment 22. The anti-CD3 antibody of any one of embodiments 1, 4, 6-9, or 11, wherein said heavy chain variable domain comprises a serine at a position corresponding to position 33 of SEQ ID NO:8.

Embodiment 23. The anti-CD3 antibody of any one of embodiments 1, 4, 6-9, or 11, wherein said heavy chain variable domain comprises a phenylalanine, a tyrosine or a glutamine at a position corresponding to position 35 of SEQ ID NO:8.

Embodiment 24. The anti-CD3 antibody of any one of embodiments 22 or 23, wherein said antibody does not bind to CD3.

Embodiment 25. A recombinant protein comprising: (i) a first antibody region capable of binding a target cell ligand; and (ii) a second antibody region, comprising: (a) a second light chain variable domain comprising a CDR L1 as set forth in SEQ ID NO:1, a CDR L2 as set forth in SEQ ID NO:2 and a CDR L3 as set forth in SEQ ID NO:3; and (b) a second heavy chain variable domain comprising a CDR H1 as set forth in SEQ ID NO:4, a CDR H2 as set forth in SEQ ID NO:5, and a CDR H3 as set forth in SEQ ID NO: 6.

Embodiment 26. A recombinant protein comprising: (i) a first antibody region capable of binding a target cell ligand; and (ii) a second antibody region, comprising: (a) a second light chain variable domain comprising a CDR L1 as set forth in SEQ ID NO:1, a CDR L2 as set forth in SEQ ID NO:2 and a CDR L3 as set forth in SEQ ID NO:3; and (b) a second heavy chain variable domain comprising a CDR H1 as set forth in SEQ ID NO:4, a CDR H2 as set forth in SEQ ID NO:16, and a CDR H3 as set forth in SEQ ID NO: 6.

Embodiment 27. A recombinant protein comprising: (i) a first antibody region capable of binding a target cell ligand; and (ii) a second antibody region, comprising: (a) a second light chain variable domain comprising a CDR L1 as set forth in SEQ ID NO:1, a CDR L2 as set forth in SEQ ID NO:2 and a CDR L3 as set forth in SEQ ID NO:3; and (b) a second heavy chain variable domain comprising a CDR H1 as set forth in SEQ ID NO:4, a CDR H2 as set forth in SEQ ID NO:19, and a CDR H3 as set forth in SEQ ID NO:20.

Embodiment 28. The recombinant protein of any one of embodiments 25-27, wherein said second light chain variable domain comprises the sequence of SEQ ID NO:7, SEQ ID NO:9, or SEQ ID NO:11.

Embodiment 29. The recombinant protein of any one of embodiments 25-27, wherein said second heavy chain variable domain comprises the sequence of SEQ ID NO:8, SEQ ID NO:10, SEQ ID NO:12, or SEQ ID NO:17.

Embodiment 30. The recombinant protein of embodiment 25, wherein said second heavy chain variable domain comprises a serine at a position corresponding to position 53 of SEQ ID NO:8.

Embodiment 31. The recombinant protein of any one of embodiments 25-29, wherein said second light chain variable domain comprises the sequence of SEQ ID NO:7, SEQ ID NO:9, or SEQ ID NO:11; and wherein said second heavy chain variable domain comprises the sequence of SEQ ID NO:8, SEQ ID NO: 10, SEQ ID NO: 12, or SEQ ID NO:17.

Embodiment 32. The recombinant protein of any one of embodiments 25, 28, 29, or 31, wherein said second light chain variable domain comprises the sequence of SEQ ID NO:7 and said second heavy chain variable domain comprises the sequence of SEQ ID NO:8.

Embodiment 33. The recombinant protein of any one of embodiments 26, 28, 29, or 31, wherein said second light chain variable domain comprises the sequence of SEQ ID NO:9 and said second heavy chain variable domain comprises the sequence of SEQ ID NO:10.

Embodiment 34. The recombinant protein of any one of embodiments 27, 28, 29, or 31, wherein said second light chain variable domain comprises the sequence of SEQ ID NO:11 and said second heavy chain variable domain comprises the sequence of SEQ ID NO:12.

Embodiment 35. The recombinant protein of any one of embodiments 25, 28, 29, or 31, wherein said second light chain variable domain comprises the sequence of SEQ ID NO:7 and said second heavy chain variable domain comprises the sequence of SEQ ID NO:17.

Embodiment 36. The recombinant protein of any one of embodiments 25-35, wherein said first antibody region is bound to said target cell ligand.

Embodiment 37. The recombinant protein of any one of embodiments 25-36, wherein said target cell ligand is a cancer protein or fragment thereof.

Embodiment 38. The recombinant protein of any one of embodiments 25-37, wherein said target cell ligand forms part of a cell.

Embodiment 39. The recombinant protein of embodiment 38, wherein said cell is a cancer cell.

Embodiment 40. The recombinant protein of any one of embodiments 25-39, wherein said second antibody region is bound to a CD3 protein.

Embodiment 41. The recombinant protein of embodiment 40, wherein said CD3 protein is a human CD3 protein.

Embodiment 42. The recombinant protein of embodiment 40, wherein said CD3 protein is a non-human primate CD3 protein.

Embodiment 43. The recombinant protein of any one of embodiments 40-42, wherein said CD3 protein forms part of a cell.

Embodiment 44. The recombinant protein of any one of embodiments 40-43, wherein said CD3 protein is expressed on the surface of a cell.

Embodiment 45. The recombinant protein of embodiment 44, wherein said cell is a T cell.

Embodiment 46. The recombinant protein of any one of embodiments 25, 29, 31, 32 or 36-39, wherein said second heavy chain variable domain comprises a serine at a position corresponding to position 33 of SEQ ID NO:8.

Embodiment 47. The recombinant protein of any one of embodiments 25, 29, 31, 32 or 36-39, wherein said second heavy chain variable domain comprises a phenylalanine, a tyrosine or a glutamine at a position corresponding to position 35 of SEQ ID NO:8.

Embodiment 48. The recombinant protein of any one of embodiments 46 or 47, wherein said second antibody region does not bind to CD3.

Embodiment 49. The recombinant protein of any one of embodiments 25-48, wherein said recombinant protein is bound to a therapeutic moiety or a diagnostic moiety.

Embodiment 50. The recombinant protein of any one of embodiments 25-49, wherein said first antibody region is a first Fab′ fragment or said second antibody region is a second Fab′ fragment.

Embodiment 51. The recombinant protein of any one of embodiments 25-49, wherein said first antibody region is a single chain variable fragment (scFv) or said second antibody region is a second single chain variable fragment (scFv).

Embodiment 52. A pharmaceutical composition comprising (i) a therapeutically effective amount of an anti-CD3 antibody of any one of embodiments 1-21, or a therapeutically effective amount of a recombinant protein of any one of embodiments 25-45 or 49-51 and (ii) a pharmaceutically acceptable excipient.

Embodiment 53. A method of treating a disease in a subject in need thereof, said method comprising administering to a subject a therapeutically effective amount of an anti-CD3 antibody of any one of embodiments 1-21 or a therapeutically effective amount of a recombinant protein of any one of embodiments 25-45 or 49-51, thereby treating said disease in said subject.

Embodiment 54. The method of embodiment 53, wherein said disease is cancer.

Embodiment 55. An isolated nucleic acid encoding an anti-CD3 antibody of any one of embodiments 1-24.

Embodiment 56. A cell comprising an anti-CD3 antibody of any one of embodiments 1-24.

Embodiment 57. A cell comprising a recombinant protein of any one of embodiments 25-51.

Embodiment 58. A method of forming an anti-CD3 antibody, said method comprising administering a polypeptide comprising the sequence of SEQ ID NO:14 or SEQ ID NO:15 to a mammal, thereby forming an anti-CD3 antibody.

Embodiment 59. The method of embodiment 58, further comprising administering an adjuvant to said mammal.

What is claimed is:

1. An anti-CD3 antibody comprising a light chain variable domain and a heavy chain variable domain,
wherein said light chain variable domain comprises:
a CDR L1 as set forth in SEQ ID NO:1, a CDR L2 as set forth in SEQ ID NO:2 and a CDR L3 as set forth in SEQ ID NO:3; and
wherein said heavy chain variable domain comprises:
a CDR H1 as set forth in SEQ ID NO: 4, a CDR H2 as set forth in SEQ ID NO:5, and a CDR H3 as set forth in SEQ ID NO: 6.

2. An anti-CD3 antibody comprising a light chain variable domain and a heavy chain variable domain,
wherein said light chain variable domain comprises:
a CDR L1 as set forth in SEQ ID NO:1, a CDR L2 as set forth in SEQ ID NO:2 and a CDR L3 as set forth in SEQ ID NO:3; and
wherein said heavy chain variable domain comprises:
a CDR H1 as set forth in SEQ ID NO: 4, a CDR H2 as set forth in SEQ ID NO:16, and a CDR H3 as set forth in SEQ ID NO: 6.

3. An anti-CD3 antibody comprising a light chain variable domain and a heavy chain variable domain,
wherein said light chain variable domain comprises:
a CDR L1 as set forth in SEQ ID NO:1, a CDR L2 as set forth in SEQ ID NO:2 and a CDR L3 as set forth in SEQ ID NO:3; and
wherein said heavy chain variable domain comprises:
a CDR H1 as set forth in SEQ ID NO:4, a CDR H2 as set forth in SEQ ID NO:19, and a CDR H3 as set forth in SEQ ID NO:20.

4. The anti-CD3 antibody of any one of claims 1-3, wherein said antibody is a humanized antibody.

5. The anti-CD3 antibody of any one of claims 1-3, wherein said antibody is a chimeric antibody.

6. The anti-CD3 antibody of any one of claims 1-3, wherein said antibody is a Fab′ fragment.

7. The anti-CD3 antibody of any one of claims 1-3, wherein said antibody is a single chain antibody (scFv).

8. The anti-CD3 antibody of any one of claims 1-3, wherein said light chain variable domain comprises the sequence of SEQ ID NO:7, SEQ ID NO:9, or SEQ ID NO:11.

9. The anti-CD3 antibody of any one of claims 1-3, wherein said heavy chain variable domain comprises the sequence of SEQ ID NO:8, SEQ ID NO:10, SEQ ID NO:12, or SEQ ID NO: 17.

10. The anti-CD3 antibody of claim 1, wherein said heavy chain variable domain comprises a serine at a position corresponding to position 53 of SEQ ID NO:8.

11. The anti-CD3 antibody of claim 1, wherein said light chain variable domain comprises the sequence of SEQ ID NO:7 and said heavy chain variable domain comprises the sequence of SEQ ID NO:8

12. The anti-CD3 antibody of claim 1, wherein said light chain variable domain comprises the sequence of SEQ ID NO:9 and said heavy chain variable domain comprises the sequence of SEQ ID NO:10

13. The anti-CD3 antibody of claim 2, wherein said light chain variable domain comprises the sequence of SEQ ID NO:7 and said heavy chain variable domain comprises the sequence of SEQ ID NO: 17.

14. The anti-CD3 antibody of claim 3, wherein said light chain variable domain comprises the sequence of SEQ ID NO:11 and said heavy chain variable domain comprises the sequence of SEQ ID NO: 12.

15. The anti-CD3 antibody of any one of claims 1-3, wherein said antibody is bound to a CD3 protein.

16. The anti-CD3 antibody of claim 15, wherein said CD3 protein is a human CD3 protein.

17. The anti-CD3 antibody of claim 15, wherein said CD3 protein is a non-human primate CD3 protein.

18. The anti-CD3 antibody of claim 15, wherein said CD3 protein forms part of a cell.

19. The anti-CD3 antibody of claim 18, wherein said CD3 protein is expressed on the surface of said cell.

20. The anti-CD3 antibody of claim 18, wherein said cell is a T cell.

21. The anti-CD3 antibody of any one of claims 1-3, wherein said antibody is bound to a therapeutic moiety or a diagnostic moiety.

22. The anti-CD3 antibody of claim 1, wherein said heavy chain variable domain comprises a serine at a position corresponding to position 33 of SEQ ID NO:8.

23. The anti-CD3 antibody of claim 1, wherein said heavy chain variable domain comprises a phenylalanine, a tyrosine or a glutamine at a position corresponding to position 35 of SEQ ID NO:8.

24. The anti-CD3 antibody of claim 22 or 23, wherein said antibody does not bind to CD3.

25. A recombinant protein comprising:
(i) a first antibody region capable of binding a target cell ligand; and
(ii) a second antibody region, comprising:
(a) a second light chain variable domain comprising a CDR L1 as set forth in SEQ ID NO:1, a CDR L2 as set forth in SEQ ID NO:2 and a CDR L3 as set forth in SEQ ID NO:3;
and
(b) a second heavy chain variable domain comprising a CDR H1 as set forth in SEQ ID NO:4, a CDR H2 as set forth in SEQ ID NO:5, and a CDR H3 as set forth in SEQ ID NO:6.

26. A recombinant protein comprising:
(i) a first antibody region capable of binding a target cell ligand; and
(ii) a second antibody region, comprising:
(a) a second light chain variable domain comprising a CDR L1 as set forth in SEQ ID NO:1, a CDR L2 as set forth in SEQ ID NO:2 and a CDR L3 as set forth in SEQ ID NO:3;
and
(b) a second heavy chain variable domain comprising a CDR H1 as set forth in SEQ ID NO:4, a CDR H2 as set forth in SEQ ID NO:16, and a CDR H3 as set forth in SEQ ID NO:6.

27. A recombinant protein comprising:
(i) a first antibody region capable of binding a target cell ligand; and
(ii) a second antibody region, comprising:
(a) a second light chain variable domain comprising a CDR L1 as set forth in SEQ ID NO:1, a CDR L2 as set forth in SEQ ID NO:2 and a CDR L3 as set forth in SEQ ID NO:3;
and
(b) a second heavy chain variable domain comprising a CDR H1 as set forth in SEQ ID NO:4, a CDR H2 as set forth in SEQ ID NO:19, and a CDR H3 as set forth in SEQ ID NO:20.

28. The recombinant protein of any one of claims 25-27, wherein said second light chain variable domain comprises the sequence of SEQ ID NO:7, SEQ ID NO:9, or SEQ ID NO:11.

29. The recombinant protein of any one of claims 25-27, wherein said second heavy chain variable domain comprises the sequence of SEQ ID NO:8, SEQ ID NO:10, SEQ ID NO:12, or SEQ ID NO:17.

30. The recombinant protein of claim 25, wherein said second heavy chain variable domain comprises a serine at a position corresponding to position 53 of SEQ ID NO:8.

31. The recombinant protein of any one of claims 25-27, wherein said second light chain variable domain comprises the sequence of SEQ ID NO:7, SEQ ID NO:9, or SEQ ID NO:11;
and
wherein said second heavy chain variable domain comprises the sequence of SEQ ID NO:8, SEQ ID NO:10, SEQ ID NO:12, or SEQ ID NO:17.

32. The recombinant protein of claim 25, wherein said second light chain variable domain comprises the sequence of SEQ ID NO:7 and said second heavy chain variable domain comprises the sequence of SEQ ID NO:8.

33. The recombinant protein of claim 26, wherein said second light chain variable domain comprises the sequence of SEQ ID NO:9 and said second heavy chain variable domain comprises the sequence of SEQ ID NO:10.

34. The recombinant protein of claim 27, wherein said second light chain variable domain comprises the sequence of SEQ ID NO: 11 and said second heavy chain variable domain comprises the sequence of SEQ ID NO: 12.

35. The recombinant protein of claim 25, wherein said second light chain variable domain comprises the sequence of SEQ ID NO:7 and said second heavy chain variable domain comprises the sequence of SEQ ID NO:17.

36. The recombinant protein of any one of claims 25-27, wherein said first antibody region is bound to said target cell ligand.

37. The recombinant protein of any one of claims 25-27, wherein said target cell ligand is a cancer protein or fragment thereof.

38. The recombinant protein of any one of claims 25-27, wherein said target cell ligand forms part of a cell.

39. The recombinant protein of claim 38, wherein said cell is a cancer cell.

40. The recombinant protein of any one of claims 25-27, wherein said second antibody region is bound to a CD3 protein.

41. The recombinant protein of claim 40, wherein said CD3 protein is a human CD3 protein.

42. The recombinant protein of claim 40, wherein said CD3 protein is a non-human primate CD3 protein.

43. The recombinant protein of claim 40, wherein said CD3 protein forms part of a cell.

44. The recombinant protein of claim 40, wherein said CD3 protein is expressed on the surface of a cell.

45. The recombinant protein of claim 44, wherein said cell is a T cell.

46. The recombinant protein of claim 25, wherein said second heavy chain variable domain comprises a serine at a position corresponding to position 33 of SEQ ID NO:8.

47. The recombinant protein of claim 25, wherein said second heavy chain variable domain comprises a phenylalanine, a tyrosine or a glutamine at a position corresponding to position 35 of SEQ ID NO:8.

48. The recombinant protein of claim 46 or 47, wherein said second antibody region does not bind to CD3.

49. The recombinant protein of any one of claims 25-27, wherein said recombinant protein is bound to a therapeutic moiety or a diagnostic moiety.

50. The recombinant protein of any one of claims 25-27, wherein said first antibody region is a first Fab′ fragment or said second antibody region is a second Fab′ fragment.

51. The recombinant protein of any one of claims 25-27, wherein said first antibody region is a single chain variable fragment (scFv) or said second antibody region is a second single chain variable fragment (scFv).

52. A pharmaceutical composition comprising (i) a therapeutically effective amount of an anti-CD3 antibody of any one of claims 1-3, or a therapeutically effective amount of a recombinant protein of any one of claims 25-27 and (ii) a pharmaceutically acceptable excipient.

53. A method of treating a disease in a subject in need thereof, said method comprising administering to a subject a therapeutically effective amount of an anti-CD3 antibody of any one of claims 1-3 or a therapeutically effective amount of a recombinant protein of any one of claims 25-27, thereby treating said disease in said subject.

54. The method of claim 53, wherein said disease is cancer.

55. An isolated nucleic acid encoding an anti-CD3 antibody of any one of claims 1-24.

56. A cell comprising an anti-CD3 antibody of any one of claims 1-3.

57. A cell comprising a recombinant protein of any one of claims 25-27.

58. A method of forming an anti-CD3 antibody, said method comprising administering a polypeptide comprising the sequence of SEQ ID NO:14 or SEQ ID NO:15 to a mammal, thereby forming an anti-CD3 antibody.

59. The method of claim 58, further comprising administering an adjuvant to said mammal.
